(19) United States
(12) Patent Application Publication
(10) Pub. No.: US 2020/0325154 A1
(43) Pub. Date: Oct. 15, 2020

(54) ANTIBACTERIAL BIAROMATIC DERIVATIVES WITH OXETANE-3-YLOXY SUBSTITUTION

(71) Applicant: IDORSIA PHARMACEUTICALS LTD.

(72) Inventors: Sylvaine CREN, Allschwil (CH); Georg RUEEDI, Allschwil (CH); Cornelia ZUMBRUNN, Allschwil (CH)

(21) Appl. No.: 16/093,954

(22) PCT Filed: Apr. 13, 2017
(86) PCT No.: PCT/IB2017/052133

§ 371 Date: Oct. 15, 2018

(30) Foreign Application Priority Data
Apr. 14, 2016 (IB) PCT/IB2016/052116

(51) Int. Cl. C07D 498/04; C07D 417/14; C07D 513/04; A61P 31/04

(52) U.S. Cl. CPC C07D 498/04; A61P 31/04; C07D 513/04; C07D 417/14

(57) ABSTRACT

The invention relates to antibacterial compounds of formula I

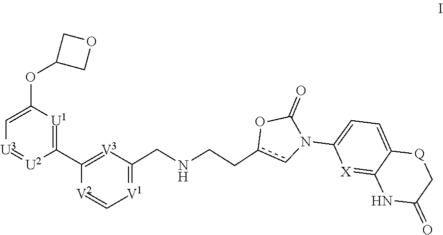

wherein U¹ represents N or CH, U² represents N or CH, U³ represents N or CH, it being understood that at most two of U¹, U², U³ can represent N at the same time;
V¹ represents N or CH, V² represents N, CH or C(OH) and V³ represents N, CH or C(OH), it being understood that at most two of V¹, V² and V³ can represent N at the same time; the dotted line “ [symbol] ” represents a bond or is absent; X represents CH or N; and Q represents O or S. It further relates pharmaceutical compositions containing these compounds and the uses of these compounds in the manufacture of medicaments for the treatment of bacterial infections. These compounds are useful antimicrobial agents effective against a variety of human and veterinary pathogens including among others Gram-positive and Gram-negative aerobic and anaerobic bacteria.

The present invention concerns antibacterial biaromatic derivatives with oxetan-3-yloxy substitution, pharmaceutical compositions containing them and uses of these compounds in the manufacture of medicaments for the treatment of bacterial infections. These compounds are useful antimicrobial agents effective against a variety of human and veterinary pathogens including among others Gram-positive and Gram-negative aerobic and anaerobic bacteria and especially against resistant strains of Pseudomonas aeruginosa and Enterobacteriaceae such as Klebsiella pneumoniae.

The intensive use of antibiotics has exerted a selective evolutionary pressure on microorganisms to produce genetically based resistance mechanisms. Modern medicine and socio-economic behaviour exacerbate the problem of resistance development by creating slow growth situations for pathogenic microbes, e.g. in artificial joints, and by supporting long-term host reservoirs, e.g. in immune-compromised patients.

In hospital settings, an increasing number of strains of Staphylococcus aureus, Streptococcus pneumoniae, Enterococcus spp., Enterobacteriacea and Pseudomonas aeruginosa, major sources of infections, are becoming multi-drug resistant and therefore difficult if not impossible to treat:
S. aureus is resistant to β-lactams, quinolones and now even to vancomycin;
S. pneumoniae is becoming resistant to penicillin or quinolone antibiotics and even to new macrolides;
Enteroccocci are quinolone and vancomycin resistant and β-lactam antibiotics are inefficacious against these strains;
Enterobacteriacea are cephalosporin and quinolone resistant and carbapenems are losing their efficacy (e.g. carbapenem-resistant K. pneumoniae);
P. aeruginosa is β-lactam and quinolone resistant.

Furthermore, the incidence of multi-drug-resistant Gram-negative strains such as Enterobacteriacae and Pseudomonas aeruginosa, is steadily increasing and new emerging organisms like Acinetobacter spp. or Clostridium difficile, which have been selected during therapy with the currently used antibiotics, are becoming a real problem in hospital settings (S. L. Solomon et al., Antibiotic Resistance Threats In the United States: Stepping Back from the Brink, Academy of Family Physician, page 940 Volume 89, Number 12, Jun. 15, 2014). Therefore, there is a high medical need for new antibacterial agents which overcome these multidrug-resistant bacilli especially Pseudomonas aeruginosa and Enterobacteriacae such as K. pneumoniae.

WO 2014/170821 describes antibacterial compounds of formula (A1)

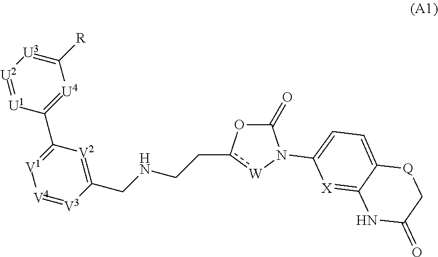

wherein

R is H, cyano, (C₁-C₃)alkoxy, cyanomethoxy, (C₃-C₆)cycloalkylmethoxy, hydroxy(C₂-C₄)alkoxy, (C₁-C₃)alkoxy-(C₂-C₃)alkoxy, (C₁-C₄)alkoxycarbonyl, 2-ethoxy-2-oxoethoxy, 2-(methylamino)-2-oxoethoxy, (1-cyanocyclobutyl)methoxy, 3-hydroxy-pyrrolidin-1-yl or (3,4-dihydroxycyclopentyl)methoxy;

U¹ is N or CR¹, U² is N or CR², U³ is N or CR³ and U⁴ is N or CR⁴, it being understood that at most three of U¹, U², U³ and U⁴ can be N at the same time;

V¹ is N or CR⁵, V² is N or CR⁶, V³ is N or CR⁷ and V⁴ is N or CH, it being understood that at most two of V¹, V², V³ and V⁴ can be N at the same time;

R² is H, cyano, hydroxy or (C₁-C₃)alkoxy;

R² is H, hydroxy or (C₁-C₃)alkoxy;

R³ is H, cyano, hydroxy, (C₁-C₃)alkoxy or carboxamido;

R⁴ is H, cyano, hydroxy or (C₁-C₃)alkoxy;

R⁵ is H, hydroxy or halogen;

R⁶ is H, hydroxy or halogen;

R⁷ is H;

the dotted line “ [symbol] ” is a bond or is absent;

W is CH or N when the dotted line “ [symbol] ” is a bond, or W represents CH₂ when the dotted line “ [symbol] ” is absent;

X is CH or N; and

Q is O or S.

In a more recent, not yet published patent application, the Applicants have furthermore described antibacterial compounds of formula (A2)

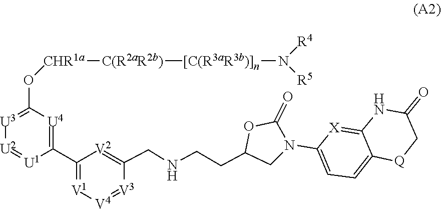

wherein

n represents 0, 1, 2 or 3;

R^{1a} represents H or (C₁-C₃)alkyl;

R^{2a} and R^{2b} independently from each other represent H or (C₁-C₃)alkyl;

R^{3a} and R^{3b} independently from each other represent H or (C₁-C₃)alkyl;

R⁴ represents H, (C₁-C₃)alkyl, or (C₂-C₃)alkyl-NR^{4a}R^{4b}, wherein R^{4a} and R^{4b} are independently from each other H or (C₁-C₃)alkyl;

R⁵ represents H, (C₁-C₃)alkyl, or (C₂-C₃)alkyl-NR^{5a}R^{5b}, wherein R^{5a} and R^{5b} are independently from each other H or (C₁-C₃)alkyl; or

R^{2a} and R^{2b} together with the carbon atom which bears them form a 3 to 6-membered cycloalkyl ring; or

R⁴ and R⁵ together with the nitrogen atom which bears them form a 4 to 6-membered heterocycloalkyl ring; or

R⁴ and R⁵ together with the nitrogen atom which bears them form a 6 to 8-membered bicyclic heterocycloalkyl ring, which bicyclic heterocycloalkyl ring may optionally be substituted by a group NR⁶R⁷, wherein R⁶ and R⁷ are independently from each other H or (C₁-C₃)alkyl; or

R⁴ and R⁵ together with the nitrogen atom which bears them and the adjacent CR^{2a}R^{2b} or CR^{3a}R^{3b} together form an amidine group; or

R^{1a} and R^{3a}, together with the carbon atoms which bear them and the carbon atom which connects these latter two atoms, form a 4 to 6-membered cycloalkyl ring, whereby R^{2a}, R^{2b} and R^{3b} each represent H, and n represents 1; or

R^{1a} and R⁴, together with the carbon and nitrogen atoms which bear them and the carbon atom(s) which connect(s) the latter two atoms, form a 4 to 6-membered heterocycloalkyl ring, whereby R^{2a}, R^{2b}, optional R^{3a} and optional R^{3b} each represent H, n represents 0 or 1, and said 4 to 6-membered heterocycloalkyl ring optionally contains a substituent selected from OCH₃ and CH₃; or

R^{2a} and R⁴ together with the carbon and nitrogen atoms which bear them and the optional carbon atom(s) which connect the latter two atoms, form a 4 to 6-membered heterocycloalkyl ring, whereby R^{1a}, optional R^{3a} and optional R^{3b} each represent H, R^{2b} represents H, NH₂ or OH, and n represents 0, 1 or 2;

U¹ represents N or CH, U² represents N, CH, C—O(C₁-C₃)alkyl, or C—CN, U³ represents N or CH and U⁴ represents N or CH, it being understood that at most three of U¹, U², U³ and U⁴ can represent N at the same time;

V¹ represents N or CH, V² represents N or CH, V³ represents N or CH and V⁴ represents N or CH, it being understood that at most three of V¹, V², V³ and V⁴ can represent N at the same time;

X represents CH or N; and

Q represents O or S.

The instant invention provides new antibacterial biaromatic derivatives based on a biphenyl or heteroaromatic biphenyl-like motif, namely the compounds of formula I described herein.

Various embodiments of the invention are presented hereafter:

1) The invention relates to compounds of formula I

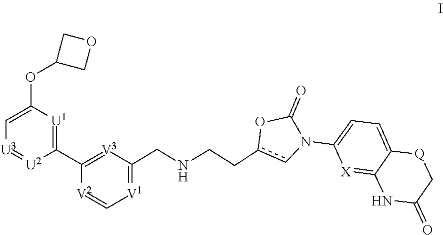

wherein

U¹ represents N or CH, U² represents N or CH, U³ represents N or CH, it being understood that at most two of U¹, U², U³ can represent N at the same time;

V¹ represents N or CH, V² represents N, CH or C(OH) and V³ represents N, CH or C(OH), it being understood that at most two of V¹, V² and V³ can represent N at the same time; the dotted line “ [symbol] ” represents a bond or is absent;

X represents CH or N; and

Q represents O or S;

and to salts (in particular pharmaceutically acceptable salts) of compounds of formula I.

The following paragraphs provide definitions of the various chemical moieties for the compounds according to the invention and are intended to apply uniformly throughout the specification and claims, unless an otherwise expressly set out definition provides a broader or narrower definition:
The term “alkyl”, used alone or in combination, refers to a straight or branched chain alkyl group containing from one to three carbon atoms. The term “(Cₓ-C_{y})alkyl” (x and y each being an integer) refers to a straight or branched chain alkyl group containing x to y carbon atoms. For example, a (C₁-C₃)alkyl group contains from one to three carbon atoms. Representative examples of alkyl groups include methyl, ethyl, propyl, and iso-propyl. Preferred are methyl and ethyl. Most preferred is methyl.
The term “quinolone-resistant”, when used in this text, refers to a bacterial strain against which ciprofloxacin has a Minimal Inhibitory Concentration of at least 16 mg/l (said Minimal Inhibitory Concentration being measured with the standard method described in “Methods for Dilution Antimicrobial Susceptibility Tests for Bacteria that Grow Aerobically”, Approved standard, 7^{th} ed., Clinical and Laboratory Standards Institute (CLSI) Document M7-A7, Wayne, Pa., USA (2006)).
The term “methicillin-resistant”, when used in this text, refers to a bacterial strain against which methicillin has a Minimal Inhibitory Concentration of at least 16 mg/l (said Minimal Inhibitory Concentration being measured with the standard method described in “Methods for Dilution Antimicrobial Susceptibility Tests for Bacteria that Grow Aerobically”, Approved standard, 7^{th} ed., Clinical and Laboratory Standards Institute (CLSI) Document M7-A7, Wayne, Pa., USA, 2006).
The term “cephalosporin-resistant”, when used in this text, refers to a bacterial strain against which cephalosporins and in particular third generation cephalosporins have a Minimal Inhibitory Concentration of at least 16 mg/l (said Minimal Inhibitory Concentration being measured with the standard method described in “Methods for Dilution Antimicrobial Susceptibility Tests for Bacteria that Grow Aerobically”, Approved standard, 7^{th} ed., Clinical and Laboratory Standards Institute (CLSI) Document M7-A7, Wayne, Pa., USA, 2006).
The term “multiresistant” when used in this text, refers to a bacterial strain which is resistant against at least two classes of established classes of antibiotics e.g. quinolones and cephalosporins.

The term “pharmaceutically acceptable salts” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example ‘Handbook of Pharmaceutical Salts. Properties, Selection and Use.’, P. Heinrich Stahl, Camille G. Wermuth (Eds.), Wiley-VCH (2008) and ‘Pharmaceutical Salts and Co-crystals’, Johan Wouters and Luc Quere (Eds.), RSC Publishing (2012).

In this text, a bond interrupted by a wavy line shows a point of attachment of the radical drawn to the rest of the molecule. For example, the radical drawn below

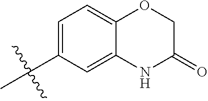 [SMILES: *c1ccc2c(c1)NC(=O)CO2]

is the 3-oxo-3,4-dihydro-2H-benzo[b][1,4]oxazin-6-yl group.

Besides, the term “room temperature” as used herein refers to a temperature of 25° C.

Unless used regarding temperatures, the term “about” placed before a numerical value “X” refers in the current application to an interval extending from X minus 10% of X to X plus 10% of X, and preferably to an interval extending from X minus 5% of X to X plus 5% of X. In the particular case of temperatures, the term “about” placed before a temperature “Y” refers in the current application to an interval extending from the temperature Y minus 10° C. to Y plus 10° C., and preferably to an interval extending from Y minus 5° C. to Y plus 5° C.

2) The invention thus notably relates to compounds of formula I that are also compounds of formula I_{CE}

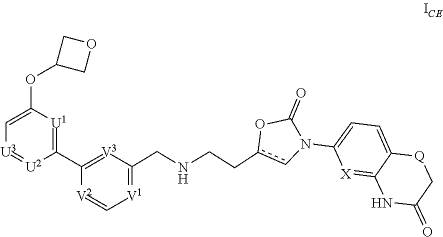

wherein

U¹ and U³ each represent N, U² represents CH, V¹ represents N and V² and V³ each represent CH, or

U¹ and U³ each represent CH, U² represents N, V¹ represents N and V² and V³ each represent CH, or

U¹ and U² each represent N, U³ represents CH, V¹ represents N and V² and V³ each represent CH, or

U¹ represents CH, U² and U³ each represent N, V¹ represents N and V² and V³ each represent CH, or

U¹ represents N and U², U³, V¹, V² and V³ each represent CH, or

U¹ and U³ each represent N, U² represents CH, V¹ and V³ each represent CH and V² represents N, or

U¹ represents CH, U² and U³ each represent N and V¹, V² and V³ each represent CH, or U¹, U², U³, V¹, V² and V³ each represent CH, or

U¹ and U³ each represent N, U² represents CH, V¹ represents N, V² represents C(OH) and V³ represents CH, or

U¹ and U² each represent N, U³ represents CH, V¹ represents N, V² represents C(OH) and V³ represents CH;

the dotted line “ [symbol] ” represents a bond or is absent;

X represents CH or N; and

Q represents O or S;

and to salts (in particular pharmaceutically acceptable salts) of compounds of formula I_{CE}.

3) A further embodiment of the invention relates to the compounds of formula I according to embodiment 1) or 2) wherein the dotted line “ [symbol] ” is absent which are also compounds of formula I_{E1}

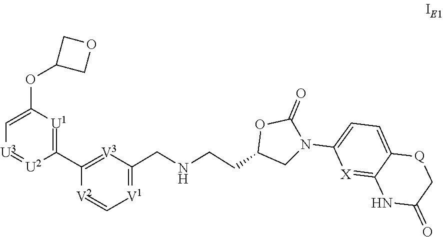

wherein the absolute configuration of the asymmetric carbon of the oxazolidinone ring is as depicted in formula I_{E1} [i.e. the absolute configuration of the asymmetric carbon of the oxazolidinone ring is (S].

4) Yet a further embodiment of the invention relates to to the compounds of formula I according to embodiment 1) or 2) wherein the dotted line “ [symbol] ” is absent which are also compounds of formula I_{E2}

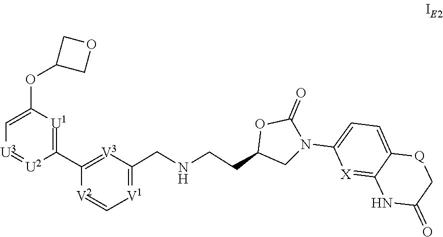

wherein the absolute configuration of the asymmetric carbon of the oxazolidinone ring is as depicted in formula I_{E2} [i.e. the absolute configuration of the asymmetric carbon of the oxazolidinone ring is (R)].

5) According to one aspect of this invention, the compounds of formula I as defined in embodiment 1) or 2) will be such that the dotted line “ [symbol] ” is absent.

6) According to the other aspect of this invention, the compounds of formula I as defined in embodiment 1) or 2) will be such that the dotted line “ [symbol] ” represents a bond.

7) According to one main variant of this invention, the compounds of formula I as defined in embodiments 1) to 6) will be such that X represents CH.

8) According to the other main variant of this invention, the compounds of formula I as defined in embodiments 1) to 6) will be such that X represents N (and notably such that X represents N and Q represents O).

9) According to one aspect of this invention, the compounds of formula I as defined in embodiments 1) to 8) will be such that

U¹ and U³ each represent N, U² represents CH, V¹ represents N and V² and V³ each represent CH, or

U¹ and U³ each represent CH, U² represents N, V¹ represents N and V² and V³ each represent CH, or

U¹ and U² each represent N, U³ represents CH, V¹ represents N and V² and V³ each represent CH, or

U¹ represents CH, U² and U³ each represent N, V¹ represents N and V² and V³ each represent CH, or

U¹ represents N and U², U³, V¹, V² and V³ each represent CH, or

U¹ and U³ each represent N, U² represents CH, V¹ and V³ each represent CH and V² represents N, or

U¹ represents CH, U² and U³ each represent N and V¹, V² and V³ each represent CH, or

U¹, U², U³, V¹, V² and V³ each represent CH; and the dotted line “ [symbol] ” is absent.

10) In particular, the compounds of formula I as defined in embodiment 9) will be such that X is N and Q is O.

11) According to another aspect of this invention, the compounds of formula I as defined in embodiments 1) to 8) will be such that

U¹ and U³ each represent N, U² represents CH, V¹ represents N, V² represents C(OH) and V³ represents CH, or

U¹ and U² each represent N, U³ represents CH, V¹ represents N, V² represents C(OH) and V³ represents CH, or

U¹ and U³ each represent N, U² represents CH, V¹ represents N, V² represents CH and V³ represents C(OH); and

the dotted line “ [symbol] ” is absent.

12) In particular, the compounds of formula I as defined in embodiment 11) will be such that X is N and Q is O.

13) One particular embodiment of this invention relates to compounds of formula I as defined in one of embodiments 1) to 8) wherein U¹ and U² each represent N, U³ represents CH, V¹ represents N, V² represents CH or C(OH) and V³ represents CH (notably to those compounds wherein U¹ and U² each represent N, U³ represents CH, V¹ represents N, V² represents C(OH) and V³ represents CH).

14) Another particular embodiment of this invention relates to compounds of formula I as defined in one of embodiments 1) to 8) wherein U¹ and U³ each represent N, U² represents CH, V¹ represents N, V² represents CH and V³ represents CH or C(OH) (notably those wherein U¹ and U³ each represent N, U² represents CH, V¹ represents N, and V² and V³ each represent CH).

15) Yet another particular embodiment of this invention relates to compounds of formula I as defined in one of embodiments 1) to 8) wherein U¹ represents N, U², U³, V¹, V² each represent CH and V³ represents CH or C(OH) (notably to those compounds wherein U¹ represents N, U², U³, V¹, V² and V³ each represent CH).

16) Preferably, the compounds of formulae I, I_{CE}, I_{E1} and I_{E2} as defined in embodiments 1) to 8) will be such that:

U¹ and U³ each represent N, U² represents CH, V¹ represents N and V² and V³ each represent CH, or

U¹ and U² each represent N, U³ represents CH, V¹ represents N and V² and V³ each represent CH, or

U¹ represents N and U², U³, V¹, V² and V³ each represent CH, or

U¹ represents CH, U² and U³ each represent N and V¹, V² and V³ each represent CH, or

U¹ and U² each represent N, U³ represents CH, V¹ represents N, V² represents C(OH) and V³ represents CH.

17) In particular, the compounds of formula I as defined in embodiment 16) will be such that X is N and Q is O.

18) More preferably, the compounds of formulae I, I_{CE}, I_{E1} and I_{E2} as defined in embodiments 1) to 4) will be such that:

U¹ and U³ each represent N, U² represents CH, V¹ represents N and V² and V³ each represent CH, or

U¹ and U² each represent N, U³ represents CH, V¹ represents N and V² and V³ each represent CH, or

U¹ represents N and U², U³, V¹, V² and V³ each represent CH, or

U¹ and U² each represent N, U³ represents CH, V¹ represents N, V² represents C(OH) and V³ represents CH; and

the dotted line “ [symbol] ” is absent.

19) In particular, the compounds of formula I as defined in embodiment 18) will be such that X is N and Q is O.

20) Preferred are the following compounds of formula I according to embodiment 1) or 2):
6-{(S)-5-[2-({4-[6-(oxetan-3-yloxy)-pyrazin-2-yl]-pyridin-2-ylmethyl}-amino)-ethyl]-2-oxo-oxazolidin-3-yl}-4H-pyrido[3,2-b][1,4]oxazin-3-one;
6-{5-[2-({4-[6-(oxetan-3-yloxy)-pyrazin-2-yl]-pyridin-2-ylmethyl}-amino)-ethyl]-2-oxo-oxazol-3-yl}-4H-pyrido[3,2-b][1,4]oxazin-3-one;
6-[(S)-5-(2-{[4-(oxetan-3-yloxy)-[2,4′]bipyridinyl-2′-ylmethyl]-amino}-ethyl)-2-oxo-oxazolidin-3-yl]-4H-pyrido[3,2-b][1,4]oxazin-3-one;
6-{(S)-5-[2-({4-[4-(oxetan-3-yloxy)-pyrimidin-2-yl]-pyridin-2-ylmethyl}-amino)-ethyl]-2-oxo-oxazolidin-3-yl}-4H-pyrido[3,2-b][1,4]oxazin-3-one;
6-{(S)-5-[2-({4-[5-(oxetan-3-yloxy)-pyridazin-3-yl]-pyridin-2-ylmethyl}-amino)-ethyl]-2-oxo-oxazolidin-3-yl}-4H-pyrido[3,2-b][1,4]oxazin-3-one;
6-[(S)-5-(2-{3-[6-(oxetan-3-yloxy)-pyridin-2-yl]-benzylamino}-ethyl)-2-oxo-oxazolidin-3-yl]-4H-pyrido[3,2-b][1,4]oxazin-3-one;
6-{(S)-5-[2-({2-[6-(oxetan-3-yloxy)-pyrazin-2-yl]-pyridin-4-ylmethyl}-amino)-ethyl]-2-oxo-oxazolidin-3-yl}-4H-pyrido[3,2-b][1,4]oxazin-3-one;
6-[(S)-5-(2-{3-[5-(oxetan-3-yloxy)-pyridazin-3-yl]-benzylamino}-ethyl)-2-oxo-oxazolidin-3-yl]-4H-pyrido[3,2-b][1,4]oxazin-3-one;
6-[(S)-5-(2-{[3′-(oxetan-3-yloxy)-biphenyl-3-ylmethyl]-amino}-ethyl)-2-oxo-oxazolidin-3-yl]-4H-pyrido[3,2-b][1,4]oxazin-3-one;
6-{(R)-5-[2-({4-[4-(oxetan-3-yloxy)-pyrimidin-2-yl]-pyridin-2-ylmethyl}-amino)-ethyl]-2-oxo-oxazolidin-3-yl}-4H-pyrido[3,2-b][1,4]oxazin-3-one;
6-{(S)-5-[2-({4-[4-(oxetan-3-yloxy)-pyrimidin-2-yl]-pyridin-2-ylmethyl}-amino)-ethyl]-2-oxo-oxazolidin-3-yl}-4H-pyrido[3,2-b][1,4]thiazin-3-one;
6-{(S)-5-[2-({4-[4-(oxetan-3-yloxy)-pyrimidin-2-yl]-pyridin-2-ylmethyl}-amino)-ethyl]-2-oxo-oxazolidin-3-yl}-4H-benzo[1,4]thiazin-3-one;
6-{5-[2-({4-[4-(oxetan-3-yloxy)-pyrimidin-2-yl]-pyridin-2-ylmethyl}-amino)-ethyl]-2-oxo-oxazol-3-yl}-4H-pyrido[3,2-b][1,4]oxazin-3-one;
6-{(S)-5-[2-({5-hydroxy-4-[6-(oxetan-3-yloxy)-pyrazin-2-yl]-pyridin-2-ylmethyl}-amino)-ethyl]-2-oxo-oxazolidin-3-yl}-4H-pyrido[3,2-b][1,4]oxazin-3-one; and
6-{(S)-5-[2-({5-hydroxy-4-[4-(oxetan-3-yloxy)-pyrimidin-2-yl]-pyridin-2-ylmethyl}-amino)-ethyl]-2-oxo-oxazolidin-3-yl}-4H-pyrido[3,2-b][1,4]oxazin-3-one; as well as the salts (in particular pharmaceutically acceptable salts) of these compounds.

The compounds of formulae I, I_{CE}, I_{E1} and I_{E2} according to the invention, i.e. according to one of embodiments 1) to 20) above, are suitable for the use as chemotherapeutic active compounds in human and veterinary medicine and as substances for preserving inorganic and organic materials in particular all types of organic materials for example polymers, lubricants, paints, fibers, leather, paper and wood.

The compounds of formulae I, I_{CE}, I_{E1} and I_{E2} according to the invention are particularly active against bacteria and bacteria-like organisms. They may therefore be particularly suitable in human and veterinary medicine for the prophylaxis and chemotherapy of local and systemic infections caused by these pathogens as well as disorders related to bacterial infections comprising pneumonia, otitis media, sinusitis, bronchitis, tonsillitis, and mastoiditis related to infection by Streptococcus pneumoniae, Moraxella catarrhalis, Staphylococcus aureus, Staphylococcus epidermidis, Staphylococcus haemolyticus, or Peptostreptococcus spp.; pharyngitis, rheumatic fever, and glomerulonephritis related to infection by Streptococcus pyogenes, Groups C and G streptococci, Corynebacterium diphtheriae, or Actinobacillus haemolyticum; respiratory tract infections related to infection by Legionella pneumophila, S. pneumonia or Chlamydia pneumoniae; blood and tissue infections, including endocarditis and osteomyelitis, caused by S. aureus, S. haemolyticus, including strains resistant to known antibacterials such as, but not limited to, beta-lactams, vancomycin, aminoglycosides, quinolones, chloramphenicol, tetracyclines and macrolides; uncomplicated skin and soft tissue infections and abscesses, and puerperal fever related to infection by S. aureus, coagulase-negative staphylococci (i.e., S. epidermidis, S. haemolyticus, etc.), S. pyogenes, Streptococcus agalactiae, Streptococcal groups C—F (minute colony streptococci), viridans streptococci, Corynebacterium minutissimum, Clostridium spp., or Bartonella henselae; uncomplicated acute urinary tract infections related to infection by S. aureus or coagulase-negative staphylococcal species; urethritis and cervicitis; sexually transmitted diseases related to infection by Chlamydia trachomatis, Haemophilus ducreyi, Treponema pallidum, Ureaplasma urealyticum, or Neiserria gonorrheae; toxin diseases related to infection by S. aureus (food poisoning and toxic shock syndrome), or Groups A, B and C streptococci; conjunctivitis, keratitis, and dacrocystitis related to infection by C. trachomatis, N. gonorrhoeae, S. aureus, S. pneumoniae, S. pyogenes or Listeria spp.

The preceding lists of infections and pathogens are to be interpreted merely as examples and in no way as limiting.

The compounds of formulae I, I_{CE}, I_{E1} and I_{E2} according to this invention, or the pharmaceutically acceptable salt thereof, may thus be used for the preparation of a medicament, and are suitable, for the prevention or treatment of a bacterial infection (notably for the prevention or treatment of a bacterial infection mediated by Staphylococcus aureus bacteria, especially for the prevention or treatment of a bacterial infection mediated by quinolone-resistant Staphylococcus aureus).

Accordingly, the compounds of formulae I, I_{CE}, I_{E1} and I_{E2} according to any one of embodiments 1) to 20), or the pharmaceutically acceptable salts thereof, may be used for the preparation of a medicament, and are suitable, for the prevention or treatment of a bacterial infection selected from the group consisting of respiratory tract infections, otitis media, meningitis, skin and soft tissue infections (whether complicated or uncomplicated), pneumonia (including hospital acquired pneumonia), bacteremia, endocarditis, intraabdominal infections, gastrointestinal infections, urinary tract infections, sexually transmitted infections, foreign body infections, osteomyelitis, Lyme disease, topical infections, or ophthalmological infections, and notably for the prevention or treatment of a bacterial infection selected from the group consisting of respiratory tract infections, otitis media, meningitis, skin and soft tissue infections (whether complicated or uncomplicated), pneumonia (including hospital acquired pneumonia) and bacteremia.

The compounds of formulae I, I_{CE}, I_{E1} and I_{E2} according to any one of embodiments 1) to 20), and the pharmaceutically acceptable salts thereof, may further be useful for the preparation of a medicament, and are suitable, for the treatment of infections that are mediated by Gram positive bacteria (such as Staphylococcus aureus, Bacillus cereus, Bacillus anthracis, Corynebacterium spp. and Propionibacterium acnes), notably by Gram positive bacteria selected from the group consisting of Bacillus cereus, Bacillus anthracis and Propionibacterium acnes. In particular, the compounds of formula I according to any one of embodiments 1) to 20), and the pharmaceutically acceptable salts thereof, can be used for the preparation of a medicament, and are suitable, for the treatment of a bacterial infection mediated by Staphylococcus aureus bacteria (especially quinolone-resistant Staphylococcus aureus bacteria).

The compounds of formulae I, I_{CE}, I_{E1} and I_{E2} according to any one of embodiments 1) to 20), and the pharmaceutically acceptable salts thereof, may further be useful for the preparation of a medicament, and are suitable, for the treatment of infections that are mediated by Gram negative bacteria (such as E. coli, Klebsiella pneumoniae and other Enterobacteriaceae, Pseudomonas aeruginosa, Stenotrophomonas maltophilia, Neisseria meningitidis, Moraxella catarrhalis and Bacteroides spp), notably by Gram negative bacteria selected from the group consisting of Escherichia coli, Klebsiella pneumoniae, Pseudomonas aeruginosa, Stenotrophomonas maltophilia, Moraxella catarrhalis and Neisseria meningitidis. In particular, the compounds of formula I according to any one of embodiments 1) to 20), and the pharmaceutically acceptable salts thereof, can be used for the preparation of a medicament, and are suitable, for the treatment of a bacterial infection mediated by Klebsiella pneumoniae bacteria (especially multiresistant or quinolone-resistant Klebsiella pneumoniae bacteria) and Pseudomonas aeruginosa.

One aspect of this invention therefore relates to the use of a compound of formulae I, I_{CE}, I_{E1} and I_{E2} according to one of embodiments 1) to 20), or of a pharmaceutically acceptable salt thereof, for the manufacture of a medicament for the prevention or treatment of a bacterial infection (in particular one of the previously mentioned infections mediated by Gram negative bacteria or one of the previously mentioned infections mediated by Gram positive bacteria). Another aspect of this invention relates to a compound of formulae I, I_{CE}, I_{E1} and I_{E2} according to one of embodiments 1) to 20), or a pharmaceutically acceptable salt thereof, for the prevention or treatment of a bacterial infection (in particular for the prevention or treatment of one of the previously mentioned infections mediated by Gram negative bacteria or of one of the previously mentioned infections mediated by Gram positive bacteria). Yet another aspect of this invention relates to a compound of formulae I, I_{CE}, I_{E1} and I_{E2} according to one of embodiments 1) to 20), or a pharmaceutically acceptable salt thereof, as a medicament. Yet a further aspect of this invention relates to a pharmaceutical composition containing, as active principle, a compound of formulae I, I_{CE}, I_{E1} and I_{E2} according to one of embodiments 1) to 20), or a pharmaceutically acceptable salt thereof, and at least one therapeutically inert excipient.

As well as in humans, bacterial infections can also be treated using compounds of formulae I, I_{CE}, I_{E1} and I_{E2} (or pharmaceutically acceptable salts thereof) in other species like pigs, ruminants, horses, dogs, cats and poultry.

The present invention also relates to pharmacologically acceptable salts and to compositions and formulations of compounds of formulae I, I_{CE}, I_{E1} and I_{E2}.

Any reference to a compound of formulae I, I_{CE}, I_{E1} and I_{E}2 in this text is to be understood as referring also to the salts (and especially the pharmaceutically acceptable salts) of such compounds, as appropriate and expedient.

A pharmaceutical composition according to the present invention contains at least one compound of formulae I, I_{CE}, I_{E1} and I_{E2} (or a pharmaceutically acceptable salt thereof) as the active agent and optionally carriers and/or diluents and/or adjuvants, and may also contain additional known antibiotics.

The compounds of formulae I, I_{CE}, I_{E1} and I_{E}2 and their pharmaceutically acceptable salts can be used as medicaments, e.g. in the form of pharmaceutical compositions for enteral or parenteral administration.

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of formula I or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

Another aspect of the invention concerns a method for the prevention or the treatment of a bacterial infection in a patient, comprising the administration to said patient of a pharmaceutically active amount of a compound of formula I according to one of embodiments 1) to 20) or a pharmaceutically acceptable salt thereof. Accordingly, the invention provides a method for the prevention or the treatment of a bacterial infection mediated by Gram negative bacteria (in particular a bacterial infection mediated by Klebsiella pneumonia bacteria, and especially by multiresistant or quinolone-resistant Klebsiella pneumonia bacteria and Pseudomonas aeruginosa bacteria) in a patient, comprising the administration to said patient of a pharmaceutically active amount of a compound of formula I according to one of embodiments 1) to 20) or a pharmaceutically acceptable salt thereof. The invention further provides a method for the prevention or the treatment of a bacterial infection mediated by Gram positive bacteria (in particular a bacterial infection mediated by Staphylococcus aureus bacteria, especially by quinolone-resistant Staphylococcus aureus bacteria) in a patient, comprising the administration to said patient of a pharmaceutically active amount of a compound of formula I according to one of embodiments 1) to 20) or a pharmaceutically acceptable salt thereof.

Moreover, the compounds of formulae I, I_{CE}, I_{E1} and I_{E2} according to this invention may also be used for cleaning purposes, e.g. to remove pathogenic microbes and bacteria from surgical instruments, catheters and artificial implants or to make a room or an area aseptic. For such purposes, the compounds of formula I could be contained in a solution or in a spray formulation.

This invention, thus, relates to the compounds of formulae I, I_{CE}, I_{E1} and I_{E2} as defined in embodiments 1) to 4), or further limited under consideration of their respective dependencies by the characteristics of any one of embodiments 5) to 20), and to pharmaceutically acceptable salts thereof. It relates furthermore to the use of such compounds as medicaments, especially for the prevention or treatment of a bacterial infection, in particular for the prevention or treatment of a bacterial infection mediated by Gram positive bacteria (in particular a bacterial infection mediated by Staphylococcus aureus bacteria, especially by quinolone-resistant Staphylococcus aureus bacteria) or for the prevention or treatment of a bacterial infection mediated by Gram negative bacteria (in particular a bacterial infection mediated by Klebsiella pneumonia bacteria, and especially by multiresistant or quinolone-resistant Klebsiella pneumonia bacteria and Pseudomonas aeruginosa bacteria), and notably for the prevention or treatment of a bacterial infection mediated by quinolone-resistant Staphylococcus aureus, Klebsiella pneumonia or Pseudomonas aeruginosa bacteria. The following embodiments relating to the compounds of formulae I, I_{CE}, I_{E1} and I_{E2} according to embodiments 1) to 4) are thus possible and intended and herewith specifically disclosed in individualised form:

1, 2+1, 3+1, 3+2+1, 4+1, 4+2+1, 5+1, 5+2+1, 6+1, 6+2+1, 7+1, 7+2+1, 7+3+1, 7+3+2+1, 7+4+1, 7+4+2+1, 7+5+1, 7+5+2+1, 7+6+1, 7+6+2+1, 8+1, 8+2+1, 8+3+1, 8+3+2+1, 8+4+1, 8+4+2+1, 8+5+1, 8+5+2+1, 8+6+1, 8+6+2+1, 9+1, 9+2+1, 9+3+1, 9+3+2+1, 9+4+1, 9+4+2+1, 9+5+1, 9+5+2+1, 9+6+1, 9+6+2+1, 9+7+1, 9+7+2+1, 9+7+3+1, 9+7+3+2+1, 9+7+4+1, 9+7+4+2+1, 9+7+5+1, 9+7+5+2+1, 9+7+6+1, 9+7+6+2+1, 9+8+1, 9+8+2+1, 9+8+3+1, 9+8+3+2+1, 9+8+4+1, 9+8+4+2+1, 9+8+5+1, 9+8+5+2+1, 9+8+6+1, 9+8+6+2+1, 10+9+1, 10+9+2+1, 10+9+3+1, 10+9+3+2+1, 10+9+4+1, 10+9+4+2+1, 10+9+5+1, 10+9+5+2+1, 10+9+6+1, 10+9+6+2+1, 10+9+7+1, 10+9+7+2+1, 10+9+7+3+1, 10+9+7+3+2+1, 10+9+7+4+1, 10+9+7+4+2+1, 10+9+7+5+1, 10+9+7+5+2+1, 10+9+7+6+1, 10+9+7+6+2+1, 10+9+8+1, 10+9+8+2+1, 10+9+8+3+1, 10+9+8+3+2+1, 10+9+8+4+1, 10+9+8+4+2+1, 10+9+8+5+1, 10+9+8+5+2+1, 10+9+8+6+1, 10+9+8+6+2+1, 11+1, 11+2+1, 11+3+1, 11+3+2+1, 11+4+1, 11+4+2+1, 11+5+1, 11+5+2+1, 11+6+1, 11+6+2+1, 11+7+1, 11+7+2+1, 11+7+3+1, 11+7+3+2+1, 11+7+4+1, 11+7+4+2+1, 11+7+5+1, 11+7+5+2+1, 11+7+6+1, 11+7+6+2+1, 11+8+1, 11+8+2+1, 11+8+3+1, 11+8+3+2+1, 11+8+4+1, 11+8+4+2+1, 11+8+5+1, 11+8+5+2+1, 11+8+6+1, 11+8+6+2+1, 12+11+1, 12+11+2+1, 12+11+3+1, 12+11+3+2+1, 12+11+4+1, 12+11+4+2+1, 12+11+5+1, 12+11+5+2+1, 12+11+6+1, 12+11+6+2+1, 12+11+7+1, 12+11+7+2+1, 12+11+7+3+1, 12+11+7+3+2+1, 12+11+7+4+1, 12+11+7+4+2+1, 12+11+7+5+1, 12+11+7+5+2+1, 12+11+7+6+1, 12+11+7+6+2+1, 12+11+8+1, 12+11+8+2+1, 12+11+8+3+1, 12+11+8+3+2+1, 12+11+8+4+1, 12+11+8+4+2+1, 12+11+8+5+1, 12+11+8+5+2+1, 12+11+8+6+1, 12+11+8+6+2+1, 13+1, 13+2+1, 13+3+1, 13+3+2+1, 13+4+1, 13+4+2+1, 13+5+1, 13+5+2+1, 13+6+1, 13+6+2+1, 13+7+1, 13+7+2+1, 13+7+3+1, 13+7+3+2+1, 13+7+4+1, 13+7+4+2+1, 13+7+5+1, 13+7+5+2+1, 13+7+6+1, 13+7+6+2+1, 13+8+1, 13+8+2+1, 13+8+3+1, 13+8+3+2+1, 13+8+4+1, 13+8+4+2+1, 13+8+5+1, 13+8+5+2+1, 13+8+6+1, 13+8+6+2+1, 14+1, 14+2+1, 14+3+1, 14+3+2+1, 14+4+1, 14+4+2+1, 14+5+1, 14+5+2+1, 14+6+1, 14+6+2+1, 14+7+1, 14+7+2+1, 14+7+3+1, 14+7+3+2+1, 14+7+4+1, 14+7+4+2+1, 14+7+5+1, 14+7+5+2+1, 14+7+6+1, 14+7+6+2+1, 14+8+1, 14+8+2+1, 14+8+3+1, 14+8+3+2+1, 14+8+4+1, 14+8+4+2+1, 14+8+5+1, 14+8+5+2+1, 14+8+6+1, 14+8+6+2+1, 15+1, 15+2+1, 15+3+1, 15+3+2+1, 15+4+1, 15+4+2+1, 15+5+1, 15+5+2+1, 15+6+1, 15+6+2+1, 15+7+1, 15+7+2+1, 15+7+3+1, 15+7+3+2+1, 15+7+4+1, 15+7+4+2+1, 15+7+5+1, 15+7+5+2+1, 15+7+6+1, 15+7+6+2+1, 15+8+1, 15+8+2+1, 15+8+3+1, 15+8+3+2+1, 15+8+4+1, 15+8+4+2+1, 15+8+5+1, 15+8+5+2+1, 15+8+6+1, 15+8+6+2+1, 16+1, 16+2+1, 16+3+1, 16+3+2+1, 16+4+1, 16+4+2+1, 16+5+1, 16+5+2+1, 16+6+1, 16+6+2+1, 16+7+1, 16+7+2+1, 16+7+3+1, 16+7+3+2+1, 16+7+4+1, 16+7+4+2+1, 16+7+5+1, 16+7+5+2+1, 16+7+6+1, 16+7+6+2+1, 16+8+1, 16+8+2+1, 16+8+3+1, 16+8+3+2+1, 16+8+4+1, 16+8+4+2+1, 16+8+5+1, 16+8+5+2+1, 16+8+6+1, 16+8+6+2+1, 17+16+1, 17+16+2+1, 17+16+3+1, 17+16+3+2+1, 17+16+4+1, 17+16+4+2+1, 17+16+5+1, 17+16+5+2+1, 17+16+6+1, 17+16+6+2+1, 17+16+7+1, 17+16+7+2+1, 17+16+7+3+1, 17+16+7+3+2+1, 17+16+7+4+1, 17+16+7+4+2+1, 17+16+7+5+1, 17+16+7+5+2+1, 17+16+7+6+1, 17+16+7+6+2+1, 17+16+8+1, 17+16+8+2+1, 17+16+8+3+1, 17+16+8+3+2+1, 17+16+8+4+1, 17+16+8+4+2+1, 17+16+8+5+1, 17+16+8+5+2+1, 17+16+8+6+1, 17+16+8+6+2+1, 18+1, 18+2+1, 18+3+1, 18+3+2+1, 18+4+1, 18+4+2+1, 19+18+1, 19+18+2+1, 19+18+3+1, 19+18+3+2+1, 19+18+4+1, 19+18+4+2+1, 20+1 and 20+2+1.

In the list above, the numbers refer to the embodiments according to their numbering provided hereinabove whereas “+” indicates the dependency from another embodiment. The different individualised embodiments are separated by commas. In other words, “8+5+1” for example refers to embodiment 8) depending on embodiment 5), depending on embodiment 1), i.e. embodiment “8+5+1” corresponds to embodiment 1) further limited by the features of embodiments 5) and 8). Likewise, “9+7+3+1” refers to embodiment 9) depending mutatis mutandis on embodiments 7) and 3), depending on embodiment 1), i.e. embodiment “9+7+3+1” corresponds to embodiment 1) further limited by the features of embodiment 3), further limited by the features of embodiments 7) and 9).

The compounds of formula I can be manufactured in accordance with the present invention using the procedures described hereafter.

### Preparation of the Compounds of Formula I

### Abbreviations

The following abbreviations are used throughout the specification and the examples:
Ac acetyl
AcOH acetic acid
aq. aqueous
Bu n-butyl
BuLi n-butyllithium
CC column chromatography over silica gel
Cipro ciprofloxacin
Cy cyclohexyl
DAD diode array detection
dba dibenzylideneacetone
DCE 1,2-dichloroethane
DCM dichloromethane
DEAD diethyl azodicarboxylate
DIAD diisopropyl azodicarboxylate
DME 1,2-dimethoxyethane
DMF N,N-dimethylformamide
DMSO dimethylsulfoxide
dppf 1,1′-bis(diphenylphosphino)ferrocene
EA ethyl acetate
ELSD evaporative light scattering detector
ESI electron spray ionisation
Et ethyl
EtOH ethanol
Hept heptane
Hex hexane
HPLC high pressure liquid chromatography
HV high vacuum conditions
LC liquid chromatography
min minute(s)
Me methyl
MeCN acetonitrile
MeOH methanol
MS mass spectroscopy
NMR Nuclear Magnetic Resonance
org. organic
PCy₃ tricyclohexylphosphine
Pd/C palladium on carbon
Pd₂(dba)₃ tris(dibenzylideneacetone)dipalladium(0)
Pd(dppf)Cl₂.CH₂Cl₂ [1,1′-bis(diphenylphosphino)ferrocene]-dichloropalladium(II) dichloromethanecomplex
Pd(PPh₃)₄ tetrakis(triphenylphosphine)palladium(0)
PEPPSI™-IPr [1,3-bis(2,6-diisopropylphenyl)imidazol-2-ylidene](3-chloropyridyl)palladium(II)dichloride
Ph phenyl
PBu₃ tri(n-butyl)phosphine
PPh₃ triphenylphosphine
prep-HPLC preparative high pressure liquid chromatography
PTFE polytetrafluoroethylene
PTSA para-toluenesulfonic acid
Q-Phos pentaphenyl(di-tert-butylphosphino)ferrocene
quant. quantitative yield
rt room temperature
sat. saturated
SK-CC01-A 2′-(dimethylamino)-2-biphenylyl-palladium(II) chloride dinorbornylphosphine complex
S-Phos 2-dicyclohexylphosphino-2′,6′-dimethoxybiphenyl
TBME tert-butyl methyl ether
tBu tert-butyl
tBuXPhos 2-di-tert-butylphosphino-2′,4′,6′-triisopropylbiphenyl
TEA triethylamine
TFA trifluoroacetic acid
THF tetrahydrofuran
TLC thin layer chromatography
tR retention time

General Reaction Techniques:

General Reaction Technique 1 (Reductive Amination);

The reaction between the amine and the aldehyde or ketone is performed in a solvent system allowing the removal of the formed water through physical or chemical means (e.g. distillation of the solvent-water azeotrope or in the presence of drying agents such as molecular sieves, MgSO₄ or Na₂SO₄). Such solvent is typically toluene, Hex, THF, DCM or DCE or a mixture of solvents such as DCE/MeOH. The reaction can be catalyzed by traces of acid (usually AcOH). The intermediate imine is reduced with a suitable reducing agent (e.g. NaBH₄, NaBH₃CN, or NaBH(OAc)₃ or through hydrogenation over a noble metal catalyst such as Pd/C. The reaction is carried out between −10° C. and 110° C., preferably between 0° C. and 60° C. The reaction can also be carried out in one pot. It can also be performed in protic solvents such as MeOH or water in the presence of a picoline-borane complex (Sato et al., Tetrahedron (2004), 60, 7899-7906). Alternatively the imine intermediate can be obtained by aza-Wittig reaction between an iminophosphorane (generated in situ from an azide by reaction with PPh₃) and an aldehyde (J. Org. Chem. (2006), 71, 2839-2847 and references therein).

General Reaction Technique 2 (Removal of Acetal Protecting Groups);

The acetal derivatives dissolved in a solvent such as THF or acetone are treated between 0° C. and +70° C. under acidic conditions such as aq. AcOH, aq. TFA, CBr₄ or HCl in MeOH, MeCN or THF. Further methods to remove acetal groups are given in T. W. Greene, P. G. M. Wuts, Protecting Groups in Organic Synthesis, 3d Ed (1999), 293-329 (Publisher: John Wiley and Sons, Inc., New York, N.Y.).

General Reaction Technique 3 (Suzuki Coupling)

The aromatic halide (typically a bromide) is reacted with the required boronic acid derivative or its boronate ester equivalent (e.g. pinacol ester) in the presence of a palladium catalyst and a base such as K₂CO₃, Cs₂CO₃, K₃PO₄, tBuONa or tBuOK between 20 and 120° C. in a solvent such as toluene, EtOH, THF, dioxane, DME or DMF, usually in the presence of water (20 to 50%). Examples of typical palladium catalysts are triarylphosphine palladium complexes such as Pd(PPh₃)₄. These catalysts can also be prepared in situ from a common palladium source such as Pd(OAc)₂ or Pd₂(dba)₃ and a ligand such as trialkylphosphines (e.g. PCy₃ or P(tBu)₃), dialkylphosphinobiphenyls (e.g. S-Phos) or ferrocenylphosphines (e.g. Q-phos). Alternatively, one can use a commercially available precatalyst based on palladacycle (e.g. SK-CC01-A) or N-heterocyclic carbene complexes (e.g. PEPPSI™-IPr). The reaction can also be performed by using the corresponding aromatic triflate. Further variations of the reaction are described in Miyaura and Suzuki, Chem. Rev. (1995), 95, 2457-2483, Bellina et al., Synthesis (2004), 2419-2440, Mauger and Mignani, Aldrichimica Acta (2006), 39, 17-24, Kantchev et al., Aldrichimica Acta (2006), 39, 97-111, Fu, Acc. Chem. Res. (2008), 41, 1555-1564, and references cited therein.

General Reaction Technique 4 (Mitsunobu Reaction):

The phenol derivative is reacted with an alcohol, a phosphine such as PPh₃ or PBu₃ and an azodicarboxylate such as DIAD or DEAD in a solvent such as THF, dioxane or DCM between 0 and 50° C. Further information about Mitsunobu reactions is given in L. Kürti and B. Czakó, Strategic Applications of Named Reactions in Organic Synthesis, (2005), 294-295 (Publisher: Elsevier Academic Press, Burlington, Mass.).

General Reaction Technique 5 (Nucleophilic Aromatic Substitution);

The aryl halide or triflate is reacted with an alcohol in the presence of a base such as NaH or KOtBu at 0° C. in a dry aprotic solvent such as THF or DMF between −20° C. and +50° C.

General Preparation Methods:

### Preparation of the Compounds of Formula I

The compounds of formula I can be manufactured by the methods given below, by the methods given in the examples or by analogous methods. Optimum reaction conditions may vary with the particular reactants or solvents used, but such conditions can be determined by a person skilled in the art by routine optimization procedures.

In the general methods hereafter, if not indicated otherwise, the generic groups U¹, U², U³, V¹, V², V³, X and Q and the dotted line “ [symbol] ” are as defined for formula I. General synthetic methods used repeatedly throughout the text below are referenced to and described in the above section entitled “General reaction techniques”. In some instances certain generic groups might be incompatible with the assembly illustrated in the procedures and schemes below and so will require the use of protecting groups. The use of protecting groups is well known in the art (see for example “Protective Groups in Organic Synthesis”, T. W. Greene, P. G. M. Wuts, Wiley-Interscience, 1999).

The compounds of formula I can be obtained by reacting a compound of formula II

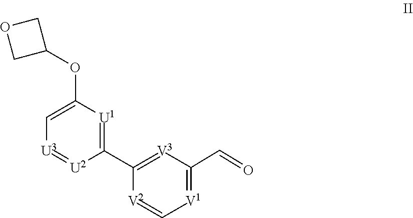

wherein U¹, U², U³, V¹, V² and V³ are as defined in formula I with a compound of formula III

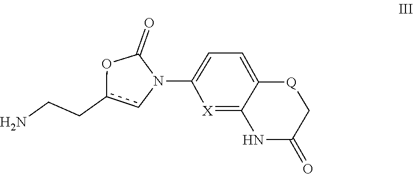

wherein X and Q are as defined in formula I using general reaction technique 1.

In the case of the preparation of compounds of formula I wherein at least one of V² and V³ represents C(OH), benzyl protected forms of the above compounds of formula II, i.e. such compounds wherein the hydroxy group(s) is (are each) replaced by benzyloxy, may be used and the resulting intermediates can then be treated under hydrogenolysis conditions over a noble metal catalyst such as Pd/C in a solvent such as MeOH/DCM to yield the compounds of formula I.

The compounds of formula I thus obtained may, if desired, be converted into their salts, and notably into their pharmaceutically acceptable salts using standard methods.

Besides, whenever the compounds of formula I are obtained in the form of mixtures of enantiomers, the enantiomers can be separated using methods known to one skilled in the art, e.g. by formation and separation of diastereomeric salts or by HPLC over a chiral stationary phase such as a Regis Whelk-O1(R,R) (10 m) column, a Daicel ChiralCel OD-H (5-10 m) column, or a Daicel ChiralPak IA (10 m) or AD-H (5 m) column. Typical conditions of chiral HPLC are an isocratic mixture of eluent A (EtOH, in the presence or absence of an amine such as TEA, diethylamine) and eluent B (Hex), at a flow rate of 0.8 to 150 ml/min.

### Preparation of the Synthesis Intermediates of Formulae II and III

Compounds of Formula II:

The compounds of formula II can be prepared as summarised in Scheme 1 hereafter.

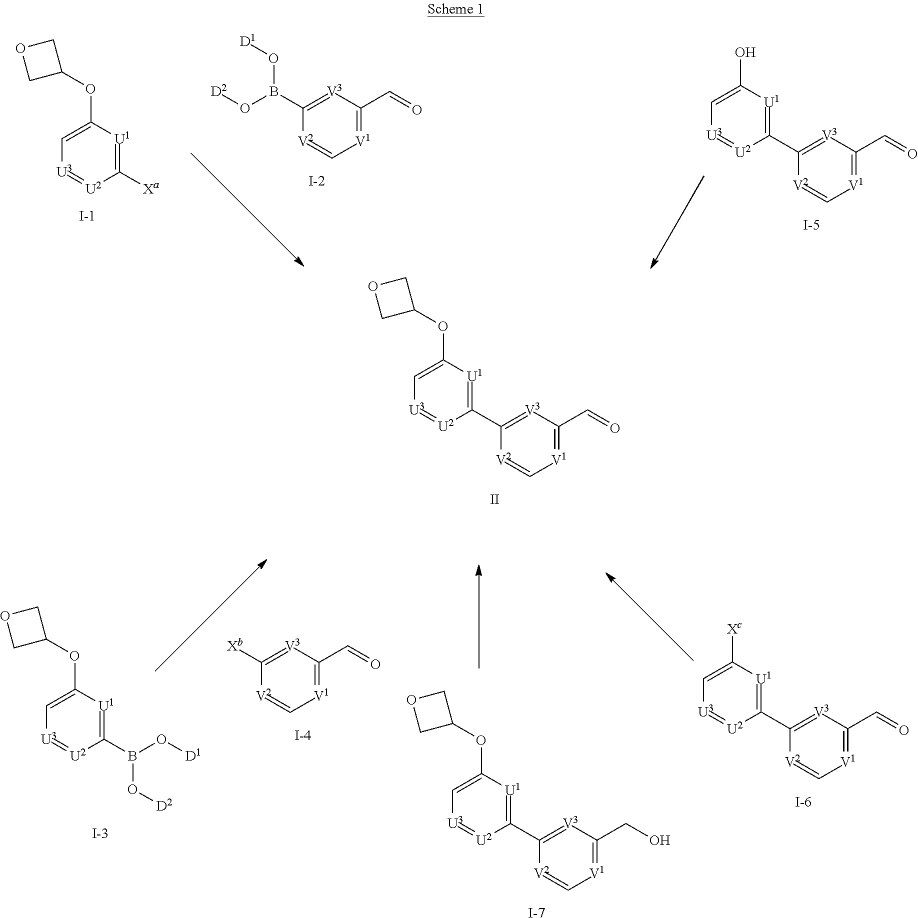

In Scheme 1, U¹, U², U³, V¹, V² and V³ are as defined in formula I, X^{a}, X^{b} and X^{c} each represent a halogen such as bromine, chlorine, iodine or a triflyl group, D¹ and D² represent H, methyl or ethyl or D¹ and D² together represent CH₂C(Me)₂CH₂ or C(Me)₂C(Me)₂.

The boronic esters or acids of formula I-2 can be reacted (Scheme 1) with the aryl halides of formula I-1 using general reaction technique 3, thus yielding the compounds of formula II. Alternatively, the latter may also be obtained by reacting the boronic esters or acids of formula I-3 with the aryl halides of formula I-4 using general reaction technique 3.

The compounds of formula II can also be obtained (Scheme 1) by reacting the compounds of formula I-5 with oxetan-3-ol using general reaction technique 4. Furthermore, the compounds of formula II can be obtained by reacting the compounds of formula I-6 with oxetan-3-ol using general reaction technique 5 or by reacting the compounds of formula I-6 with oxetan-3-ol in the presence of a base such as caesium carbonate, a palladium catalyst and a phosphine ligand in toluene between 80 and 100° C. Alternatively, the compounds of formula IT can also be obtained by reacting the compounds of formula I-7 with manganese(IV) oxide in a solvent such as MeCN between 55 and 75° C. or with other oxidants such as Dess-Martin periodinane in DCM.

Finally, the aldehyde intermediates as shown in Scheme 1 may optionally be protected as their corresponding acetals. The acetal protecting group can then be removed using general reaction technique 2.

Compounds of Formula III:

The compounds of formula III can be prepared as described in WO 2014/170821.

### Preparation of the Synthesis Intermediates of Formulae I-1, I-2, I-3, I-4, I-5, I-6 and I-7

The compounds of formulae I-1, I-2, I-3 and I-4 can be prepared as described in WO 2014/170821 or by analogous methods.

The compounds of formulae I-5, I-6 and I-7 are commercially available or can be prepared as described in the “EXAMPLES” section, in analogy thereto or by standard methods known to one skilled in the art.

Particular embodiments of the invention are described in the following Examples, which serve to illustrate the invention in more detail without limiting its scope in any way.

### EXAMPLES

All temperatures are stated in ° C. Unless otherwise indicated, the reactions take place at rt.

Analytical TLC characterizations were performed with 0.2 mm plates: Merck, Silica gel 60 F₂₅₄. Elution is performed with EA, Hept, DCM, MeOH or mixtures thereof. Detection was done with UV or with a solution of KMnO₄ (10 g), Na₂CO₃ (20 g) and H₂O (1 L) with subsequent heating.

CCs were performed using Brunschwig 60A silica gel (0.032-0.63 mm) or using an ISCO CombiFlash system and prepacked SiO₂ cartridges, elution being carried out with either Hept-EA or DCM-MeOH mixtures with an appropriate gradient.

The compounds were characterized by ¹H-NMR (400 MHz, Bruker Avance 400 or 500 MHz, Bruker Avance 500 Cryoprobe). Chemical shifts 6 are given in ppm relative to the solvent used; multiplicities: s=singlet, d=doublet, t=triplet, q=quadruplet, p=pentuplet, hex=hexet, hep=heptet, m=multiplet, br.=broad; coupling constants J are given in Hz. Alternatively compounds were characterized by LC-MS (Thermo Finnigan MSQPlus with Agilent G4220A); by TLC (TLC plates from Merck, Silica gel 60 F₂₅₄); or by melting point.

The analytical LC-MS data have been obtained using the following respective conditions:
MS1 data:
   Column: Zorbax SB-Aq, 3.5 m, 4.6×50 mm;
   Injection volume: 1 μL;
   Column oven temperature: 40° C.;
   Pump: Dionex HPG-3200RS;
   Makeup pump: Dionex ISO-3100SD;
   DAD: Dionex DAD-30000RS;
   MS: Thermo MSQ Plus;
   ELSD: Sedere Sedex 85;
   Detection: UV 210 nm, ELSD and MS;
   MS ionization mode: ESI+;
   Eluents: A: H₂O+0.04% TFA; and B: MeCN;
   Flow rate: 4.5 mL/min;
   Gradient: 5% B (0.00 min-0.01 min), 5% B to 95% B (0.01 min-1.00 min), 95% B (1.00 min-1.45 min).
MS2 data:
   Column: Zorbax RRHD SB-Aq, 3.0×50 mm, 1.8 μm;
   Injection volume: 0.30 μL;
   Column oven temperature: 40° C.;
   Pump: Agilent G4220A Binary Pump;
   Makeup pump: none;
   DAD: Agilent G4212A;
   MS: Thermo MSQ Plus;
   ELSD: Sedere Sedex 90;
   Detection: UV 210 nm, ELSD and MS;
   MS ionization mode: ESI+;
   Eluents: A: H₂O+0.04% TFA; and B: MeCN;
   Eluent flow rate: 1.6 mL/min;
   Gradient: 5% B (0.00 min-0.01 min), 5% B to 95% B (0.01 min-1.20 min), 95% B (1.20 min-1.90 min).
MS3 data:
   Column: Waters BEH C18, 3.0×50 mm, 2.5 μm;
   Eluents: A: H₂O/NH₃ (c(NH₃)=13 mmol/L; and B: MeCN;
   Otherwise same parameters as for obtaining MS2 data.
MS4 data:
   Column: Zorbax SB-Aq, 3.5 m, 4.6×50 mm;
   Injection volume: 1 μL;
   Column oven temperature: 40° C.;
   Pump: Agilent G4220A;
   Makeup pump: Dionex HPG-3200SD;
   DAD: Agilent G4212A;
   MS: Thermo MSQ Plus;
   ELSD: Sedere Sedex 90;
   Detection: UV 210 nm, ELSD and MS;
   MS ionization mode: ESI+;
   Eluents: A: H₂O+0.04% TFA; and B: MeCN;
   Flow rate: 4.5 mL/min;
   Gradient: 5% B (0.00 min-0.08 min), 5% B to 95% B (0.08 min-1.07 min), 95% B (1.07 min-1.57 min).

The number of decimals given for the corresponding [M+H⁺] peak(s) of each tested compound depends upon the accuracy of the LC-MS device actually used.

The prep-HPLC purifications were performed on a Gilson HPLC system, equipped with a Gilson LH215 autosampler, Gilson 333/334 pumps, Thermo Finnigan MSQ Plus detector system, and a Dionex UVD340U (or Dionex DAD-3000) UV detector, using the following respective conditions:
Method 1:
   Column: Waters XBridge C18, 10 μm, 30×75 mm;
   Flow rate: 75 mL/min;
   Eluents: A: H₂O+0.5% NH₄OH; B: MeCN;
   Gradient: 90% A to 5% A (0.0 min-4.0 min), 5% A (4.0 min-6.0 min).
Method 2:
   Gradient: 95% A to 50% A (0.0-3.0 min), 50% A to 5% A (3.0-4.0 min), 5% A (4.0-6.0 min);
   Otherwise same parameters as described in method 1.

The following other purification methods were furthermore used:
Filtration over Si-carbonate: silica bound equivalent of tetramethyl ammonium carbonate, SiliaPrep SPE cartridges Carbonate, 200 mg, 3 mL (Silicycle SPE-R66030B-03G).
Filtration over Alumina cartridges: polar sorbent basic character, SiliaPrep SPE Cartridges Alumina Neutral, 1 g, 6 mL (Silicycle SPE-AUT-0054-06S).

Preparations:

General Building Blocks

### Preparation BB1: 4-(4,4,5,5-tetramethyl-[1,3,2]dioxaborolan-2-yl)-pyridine-2-carbaldehyde

A mixture of 4-bromopyridine-2-carboxaldehyde (1.86 g; commercial), bis(pinacolato)-diboron (2.82 g), potassium acetate (2.48 g) and Pd(dppf)Cl₂.CH₂Cl₂ (408 mg) in dioxane (20 mL) was degassed for 5 min with N₂ and stirred at 90° C. for 2 h. The mixture was cooled down to rt, diluted with EA, filtered through a pad of Celite, concentrated under reduced pressure and used directly in the next step.

¹H NMR (CDCl₃) δ: 10.13 (s, 1H); 8.82 (d, J=4.6 Hz, 1H); 8.34 (s, 1H); 7.87 (d, J=4.4 Hz, 1H); 1.33 (m, 12H).

### Preparation BB2: 2-(2-(dimethoxymethyl)pyridin-4-yl)pyrimidin-4-ol

### BB2.i. 4-bromo-2-dimethoxymethyl-pyridine

A solution of 4-bromopyridine-2-carboxaldehyde (10 g), trimethyl orthoformate (23.3 mL) and PTSA (1.51 g) in MeOH (261 mL) was stirred at reflux for 1 day. Sat. aq. NaHCO₃ and EA were added, the layers were separated and the aq. layer was twice extracted with EA, dried over MgSO₄, filtered and concentrated under reduced pressure. The crude product was purified by filtration over 200 mL silicagel using Hept/EA 1/1 as solvent. The title compound was obtained as a colourless liquid (11.5 g; 95% yield).

MS1 (ESI, m/z): 231.99 [M+H⁺]; t_{R}=0.63 min.

### BB2.ii. Lithium salt of (2-(dimethoxymethyl)pyridin-4-yl)boric acid

An oven-dried round-bottomed flask under argon atmosphere was charged with intermediate BB2.i (11.9 g), toluene (200 mL) and THF (50 mL) and cooled to −72° C. Triisopropylborate (12.5 mL) was added dropwise, followed by dropwise addition of BuLi (21.6 mL) over 30 min, keeping the temperature below −65° C. The mixture was allowed to stir at −78° C. over night (slowly warming to rt). The resulting solution was concentrated under reduced pressure. The resulting beige solid was suspended with acetone (300 mL) and water (50 mL) and the mixture was stirred at 45° C. for 2 h. The majority of the acetone was distilled off and the residue was freeze-dried to give an orange solid (11.75 g; quant.).

¹H NMR (MeOD) δ: 8.27 (dd, J=0.8, 4.8 Hz, 1H); 7.77 (s, 1H); 7.50-7.52 (m, 1H); 5.33 (s, 1H), 4.96-4.99 (m, 6H).

### BB2.iii. 4-benzyloxy-2-(2-dimethoxymethyl-pyridin-4-yl)-pyrimidine

An oven-dried round-bottomed flask under N₂ atmosphere was charged with 4-benzyloxy-2-chloropyrimidine (1.73 g, commercial) and intermediate BB2.ii (1.44 g), K₂CO₃ (1.80 g), Pd₂(dba)₃ (307 mg) and PCy₃ (226 mg). Dioxane (25 mL) and water (6.25 mL) were added. The resulting suspension was degassed with N₂, the reaction flask was sealed and heated at 100° C. for 2.5 h. Toluene was added and the mixture was concentrated under reduced pressure. The residue was suspended with EA, the solids were filtered off and the filtrate was concentrated under reduced pressure. The title compound was obtained, after purification by CC (Combiflash; Hept to Hept/EA 1/1), as an off-white solid (1.08 g, 49% yield).

MS1 (ESI, m/z): 337.90 [M+H⁺]; t_{R}=0.83 min.

### BB2.iv. 2-(2-dimethoxymethyl-pyridin-4-yl)-pyrimidin-4-ol

To a solution of intermediate BB2.iii (50 mg) in MeOH (2 mL) and DCM (0.2 mL) under N₂ was added Pd/C (10%; 9.07 mg). The mixture was hydrogenated for 1 h. The suspension was filtered over a PTFE filter and the filtrate was concentrated under reduced pressure. The title compound was obtained, after recrystallization from DCM/TBME, as a colourless solid (660 mg; quant.).

MS1 (ESI, m/z): 248.06 [M+H⁺]; t_{R}=0.74 min.

### Preparation BB3: 2-chloro-6-(4-(dimethoxymethyl)pyridin-2-yl)pyrazine

A mixture of 2-bromo-4-(dimethoxymethyl)pyridine (116 mg, commercial), 6-chloropyrazine-2-boronic acid pinacol ester (180 mg, commercial) and aq. 2M Na₂CO₃ (0.75 mL) in dioxane (2 mL) was degassed for 10 min with N₂. Pd(dppf)Cl₂.CH₂Cl₂ (40 mg) was then added and the resulting orange suspension stirred at 85° C. (pre-heated oil bath) in a closed flask for 1 h and at rt for 2 days. The mixture was cooled down to rt, water and EA were added. The layers were separated and the aq. phase was extracted with EA (2×). The combined org. extracts were dried over MgSO₄, filtered and concentrated under reduced pressure. The crude residue was purified by CC (CombiFlash, 0-10% EA in Hept) and the title compound was obtained as a yellow oil (55 mg; 41% yield).

MS2 (ESI, m/z): 265.95 [M+H⁺]; t_{R}=0.95 min.

### Preparation BB4: rac-(5-(benzyloxy)-2-(((tetrahydro-2H-pyran-2-yl)oxy)methyl)pyridin-4-yl)-boronic acid

BB4.i. Rac-5-benzyloxy-2-(tetrahydro-pyran-2-yloxymethyl)-pyran-4-one:

To a suspension of 5-benzyloxy-2-hydroxymethyl-pyran-4-one (11.5 g; commercial) and PTSA (44.4 mg) in THF (50 mL) was added 3,4-dihydro-2H-pyran (5.59 mL; commercial) and the mixture was stirred at rt overnight. NaOH 1M (0.281 mL) was added and the mixture concentrated under reduced pressure. The title compound was obtained, after purification by CC (CombiFlash, Hept to Hept/EA 0/1), as a colourless solid (15.36 g; 98% yield).

MS1 (ESI, m/z): 317.05 [M+H⁺]; t_{R}=0.80 min.

### BB4.ii. Rac-5-benzyloxy-2-(tetrahydro-pyran-2-yloxymethyl)-1H-pyridin-4-one

A thick suspension of intermediate BB4.i (15.3 g) in EtOH (80 mL) and sat. NH₄C₁ (160 mL) was carefully and stepwise heated to 90° C. overnight. The majority of EtOH was removed under reduced pressure. The precipitate was filtered and triturated twice with TBME, filtered and dried at HV to afford the title compound as colourless crystals (12.75 g; 83% yield).

MS1 (ESI, m/z): 316.11 [M+H⁺]; t_{R}=0.66 min.

### BB4.iii. Rac-trifluoro-methanesulfonic acid 5-benzyloxy-2-(tetrahydro-pyran-2-yloxymethyl)-pyridin-4-yl ester

To a cooled suspension of intermediate BB4.ii (5.0 g) and TEA (3.31 mL) in DCM/DMF 2/1 (15.9 mL) was added N-phenyl-bis-(trifluoromethansulfonimide) (6.2 g; commercial) portionwise at 0-5° C. The mixture was allowed to warm to rt and stirred at rt for 30 min. DCM was evaporated under reduced pressure. The residue was partitioned between sat. NaHCO₃ and EA. The aq. phase was extracted with EA. The combined org. extracts were washed with water and brine, dried over MgSO₄, filtrated and concentrated under reduced pressure. The title compound was obtained, after purification by CC (CombiFlash, Hept to Hept/EA 0/1), as a colourless solid (6.10 g; 86% yield).

MS1 (ESI, m/z): 448.03 [M+H⁺]; t_{R}=1.00 min.

### BB4.iv. Rac-(5-(benzyloxy)-2-(((tetrahydro-2H-pyran-2-yl)oxy)methyl)pyridin-4-yl)-boronic acid

To a mixture of intermediate BB4.iii (500 mg), bis(pinacolato)diboron (304 mg), Pd(dppf)Cl₂.CH₂Cl₂ (45.6 mg) and KOAc (166 mg) degassed with N₂ was added dioxane (5.5 mL). The suspension was degassed with N₂ and then rapidly heated to 90° C. (pre-heated metal block). The mixture was allowed to cool to rt, diluted with TBME, filtrated and concentrated under reduced pressure to afford the title compound as a brown oi (700 mg; quant.).

MS1 (ESI, m/z): 344.07 [M+H⁺]; t_{R}=0.63 min.

### Preparation BB5: 2-(oxetan-3-yloxy)-6-(4,4,5,5-tetramethyl-1,3,2-dioxaborolan-2-yl)pyrazine

To a mixture of 2-chloro-6-(oxetan-3-yloxy)pyrazine (187 mg, commercial), bis(pinacolato)diboron (337 mg), Pd(dppf)Cl₂.CH₂Cl₂ (40 mg) and KOAc (294 mg) degassed with N₂ was added dioxane (5 mL). The suspension was degassed with N₂ and then stirred at 90° C. for 30 min. The mixture was allowed to cool to rt, diluted with EA filtered through a pad of Celite, concentrated under reduced pressure and used directly in the next step.

### EXAMPLES OF COMPOUNDS ACCORDING TO THE INVENTION

### Example 1: 6-{(S)-5-[2-({4-[6-(oxetan-3-yloxy)-pyrazin-2-yl]-pyridin-2-ylmethyl}-amino)-ethyl]-2-oxo-oxazolidin-3-yl}-4H-pyrido[3,2-b][1,4]oxazin-3-one

### 1.i. 2-bromo-6-(oxetan-3-yloxy)-pyrazine

A stirred mixture of 2,6-dibromopyrazine (200 mg, commercial) and oxetan-3-ol (64.2 mg; commercial) in THF (4 mL) was treated with NaH (33 mg) at 0° C. The mixture was allowed to stir at 0° C. for 90 min. The mixture was quenched with water and extracted twice with EA. The combined org. layers were dried over MgSO₄, filtered and concentrated under reduced pressure. The title compound was obtained, after purification by CC (CombiFlash, Hept to Hept/EA 1/1), as a colourless solid (97 mg; 51% yield).

MS1 (ESI, m/z): 231.02 [M+H⁺]; t_{R}=0.67 min.

### 1.ii. 4-[6-(oxetan-3-yloxy)-pyrazin-2-yl]-pyridine-2-carbaldehyde

To a suspension of intermediate 1.i (93 mg), the compound of Preparation BB1 (188 mg) and K₂CO₃ (167 mg) in 1,4-dioxane (2 mL) and water (0.7 mL) under N₂ was added Pd(PPh₃)₄ (23.3 mg). The mixture was degassed and flushed with N₂ and stirred in the microwave at 110° C. for 45 min. The mixture was partitioned between EA and water, the aq. layer was extracted with EA and the combined org. layers were washed with brine, dried over MgSO₄, filtered and concentrated under reduced pressure. The title compound was obtained, after purification by CC (CombiFlash, Hept to Hept/EA 1/2), as a yellow solid (79 mg; 76% yield).

MS1 (ESI, m/z): 257.89 [M+H⁺]; t_{R}=0.67 min.

### 1.iii. 6-{(S)-5-[2-({4-[6-(oxetan-3-yloxy)-pyrazin-2-yl]-pyridin-2-ylmethyl}-amino)-ethyl]-2-oxo-oxazolidin-3-yl}-4H-pyrido[3,2-b][1,4]oxazin-3-one

A solution of intermediate 1.ii (72 mg) and (S)-6-(5-(2-aminoethyl)-2-oxo-oxazolidin-3-yl)-2H-pyrido[3,2-b][1,4]oxazin-3(4H)-one (65.4 mg, prepared according to WO 2014/170821) in DCM (1.2 mL) and MeOH (1.2 mL) was treated with NaBH(OAc)₃ (142 mg) and stirred at rt for 90 min. NaHCO₃ was added and the mixture was extracted twice with DCM. The combined org. layers were dried over MgSO₄, filtered and concentrated under reduced pressure. The title compound was obtained, after purification by prep-HPLC (method 1), as an off-white solid (49 mg; 42% yield).

¹H NMR (d₆-DMSO) δ: 9.01 (s, 1H); 8.65-8.66 (m, J=5.1 Hz, 1H); 8.48 (s, 1H); 8.09 (d, 1H); 7.91 (dd, J=5.2, 1.7 Hz, 1H); 7.58 (m, 1H); 7.42 (d, J=8.7 Hz, 1H); 5.71-5.78 (m, 1H); 5.00 (m, 2H); 4.84 (m, 1H); 4.68 (dd, J=7.6, 5.3 Hz, 2H); 4.61 (s, 2H); 4.23 (m, 1H); 3.91 (s, 2H); 3.75-3.80 (m, 1H); 2.67-2.73 (m, 2H); 1.88-2.01 (m, 2H).

MS1 (ESI, m/z): 520.14 [M+H⁺]; t_{R}=0.59 min.

### Example 2: 6-{5-[2-({4-[6-(oxetan-3-yloxy)-pyrazin-2-yl]-pyridin-2-ylmethyl}-amino)-ethyl]-2-oxo-oxazol-3-yl}-4H-pyrido[3,2-b][1,4]oxazin-3-one

Starting from intermediate 1.ii (27 mg) and 6-(5-(2-aminoethyl)-2-oxo-oxazol-3(2H)-yl)-2H-pyrido[3,2-b][1,4]oxazin-3(4H)-one (27.4 mg, prepared according to WO 2014/170821) and proceeding in analogy to Example 1, step 1.iii, the title compound was obtained, after purification by prep-HPLC (method 1), as a colourless solid (28 mg; 57% yield).

MS1 (ESI, m/z): 518.12 [M+H⁺]; t_{R}=0.60 min.

### Example 3: 6-[(S)-5-(2-{[4-(oxetan-3-yloxy)-[2,4′]bipyridinyl-2′-ylmethyl]-amino}-ethyl)-2-oxo-oxazolidin-3-yl]-4H-pyrido[3,2-b][1,4]oxazin-3-one

### 3.i. 2-bromo-4-(oxetan-3-yloxy)-pyridine

To a solution of 2-bromo-4-hydroxypyridine (200 mg; commercial), oxetan-3-ol (94.1 mg) and PPh₃ (332 mg) in THF (6 mL) was added dropwise DIAD (0.18 mL). The mixture was then heated to 55° C. and stirred at this temperature for 1 day. DIAD (0.18 mL) was added again and the mixture was stirred at 55° C. for 3 days. The mixture was partitioned between EA and water, the aq. layer was extracted with EA and the combined org. layers were washed with brine, dried over MgSO₄, filtered and concentrated under reduced pressure. The title compound was obtained, after purification by CC (CombiFlash, Hept to Hept/EA 1/2), as a yellow solid (117 mg; 44% yield).

MS1 (ESI, m/z): 229.98 [M+H⁺]; t_{R}=0.61 min.

### 3.ii. 4-(oxetan-3-yloxy)-[2,4]bipyridinyl-2′-carbaldehyde

Starting from intermediate 3.i (113 mg) and the compound of Preparation BB1 (79.8 mg) and proceeding in analogy to Example 1, step 1.ii, the title compound was obtained after purification by CC (CombiFlash, Hept/EA 2/1 to EA) as an orange oil (65 mg; 61% yield).

MS1 (ESI, m/z): 257.03 [M+H⁺]; t_{R}=0.55 min.

### 3.iii. 6-[(S)-5-(2-{[4-(oxetan-3-yloxy)-[2,4′]bipyridinyl-2′-ylmethyl]-amino}-ethyl)-2-oxo-oxazolidin-3-yl]-4H-pyrido[3,2-b][1,4]oxazin-3-one

Starting from intermediate 3.ii (60 mg) and (S)-6-(5-(2-aminoethyl)-2-oxo-oxazolidin-3-yl)-2H-pyrido[3,2-b][1,4]oxazin-3(4H)-one (41 mg; prepared according to WO 2014/170821) and proceeding in analogy to Example 1, step 1.iii, the title compound was obtained, after purification by prep-HPLC (method 1), as a colourless solid (39 mg; 54% yield).

MS1 (ESI, m/z): 519.12 [M+H⁺]; t_{R}=0.54 min.

### Example 4: 6-{(S)-5-[2-({4-[4-(oxetan-3-yloxy)-pyrimidin-2-yl]-pyridin-2-ylmethyl}-amino)-ethyl]-2-oxo-oxazolidin-3-yl}-4H-pyrido[3,2-b][1,4]oxazin-3-one

### 4.i. 2-(2-dimethoxymethyl-pyridin-4-yl)-4-(oxetan-3-yloxy)-pyrimidine

To a solution of the compound of Preparation BB2 (80 mg), oxetan-3-ol (27.8 mg) and PPh₃ (93.4 mg) in THF (1 mL) was added dropwise DIAD (0.05 mL). The mixture was stirred at 50° C. for 7 h and concentrated under reduced. The title compound was obtained, after purification by prep-HPLC (method 2), as a yellow oil (53 mg; 54% yield).

MS1 (ESI, m/z): 304.03 [M+H⁺]; t_{R}=0.64 min.

### 4.ii. 4-[4-(oxetan-3-yloxy)-pyrimidin-2-yl]-pyridine-2-carbaldehyde

Intermediate 4.i (55 mg) was dissolved in water (1 mL) and THF (1 mL), PTSA (7.04 mg) was added and the mixture was heated to 60° C. for 1 day. The mixture was concentrated under reduced pressure and dried at HV for 5 min, to afford the title compound as a yellowish oil (60 mg; quant.).

MS3 (ESI, m/z): 257.98 [M+H⁺]; t_{R}=0.69 min.

### 4.iii. 6-{(S)-5-[2-({4-[4-(oxetan-3-yloxy)-pyrimidin-2-yl]-pyridin-2-ylmethyl}-amino)-ethyl]-2-oxo-oxazolidin-3-yl}-4H-pyrido[3,2-b][1,4]oxazin-3-one

This compound was obtained starting from intermediate 4.ii (46.6 mg) and (S)-6-(5-(2-aminoethyl)-2-oxo-oxazolidin-3-yl)-2H-pyrido[3,2-b][1,4]oxazin-3(4H)-one (42.8 mg; prepared according to WO 2014/170821) and proceeding in analogy to Example 1, step 1.iii. The crude product was purified by CC (CombiFlash, DCM to DCM/MeOH 9/1+1% NH₄OH) followed by trituration with MeCN/25% aq. NH₄OH/TBME, filtered and dried at HV to afford a colourless solid (26 mg; 28% yield).

MS1 (ESI, m/z): 520.10 [M+H⁺]; t_{R}=0.60 min.

### Example 5: 6-{(S)-5-[2-({4-[5-(oxetan-3-yloxy)-pyridazin-3-yl]-pyridin-2-ylmethyl}-amino)-ethyl]-2-oxo-oxazolidin-3-yl}-4H-pyrido[3,2-b][1,4]oxazin-3-one

### 5.i. 3-chloro-5-(oxetan-3-yloxy)-pyridazine

To an ice-cold suspension of NaH (96 mg) in THF (3 mL) was added a solution of oxetan-3-ol (172 mg) in THF (2 mL). The resulting grey suspension was stirred at 0° C. for 30 min. 3,5-dichloropyridazine (301 mg) was then added in one portion and the resulting yellow suspension was stirred at rt for 1 h. Water was added and the mixture extracted with EA (3×). The combined org. extracts were dried over MgSO₄, filtered and concentrated under reduced pressure. The title compound was obtained, after purification by CC (CombiFlash, Hept to Hept/EA 1:1), as a colourless solid (232 mg; 62% yield).

MS4 (ESI, m/z): 187.25 [M+H⁺]; t_{R}=0.49 min.

### 5.ii. 4-[5-(oxetan-3-yloxy)-pyridazin-3-yl]-pyridine-2-carbaldehyde

A mixture of the compound of Preparation BB1 (466 mg), intermediate 5.i (232 mg), K₂CO₃ (292 mg), PCy₃ (43.1 mg) and Pd₂(dba)₃ (56.9 mg) in dioxane (5 mL) and water (1 mL) was degassed with N₂ for 10 min. The resulting brown suspension was stirred at 85° C. for 15 h in a sealed flask. The mixture was diluted with EA, filtered through a glass fibre filter and concentrated under reduced pressure. The title compound was obtained, after purification by CC (Combiflash; Hept to Hept/EA 1/1), as an orange solid (216 mg; 68% yield).

MS3 (ESI, m/z): 257.87 [M+H⁺]; t_{R}=0.51 min.

### 5.iii. 6-{(S)-5-[2-({4-[5-(oxetan-3-yloxy)-pyridazin-3-yl]-pyridin-2-ylmethyl}-amino)-ethyl]-2-oxo-oxazolidin-3-yl}-4H-pyrido[3,2-b][1,4]oxazin-3-one

Starting from intermediate 5.ii (216 mg) and (S)-6-(5-(2-aminoethyl)-2-oxo-oxazolidin-3-yl)-2H-pyrido[3,2-b][1,4]oxazin-3(4H)-one (234 mg; prepared according to WO 2014/170821) and proceeding in analogy to Example 1, step 1.iii, the title compound was obtained, after purification by CC (CombiFlash, DCM to DCM/[1% NH₃ in MeOH]9/1), as a pale yellow foam (275 mg; 63% yield).

MS1 (ESI, m/z): 520.05 [M+H⁺]; t_{R}=0.61 min.

### Example 6: 6-[(S)-5-(2-{3-[6-(oxetan-3-yloxy)-pyridin-2-yl]-benzylamino}-ethyl)-2-oxo-oxazolidin-3-yl]-4H-pyrido[3,2-b][1,4]oxazin-3-one

### 6i. 2-bromo-6-(oxetan-3-yloxy)-pyridine

A stirred mixture of 2,6-dibromopyridine (300 mg, commercial) and oxetan-3-ol (96.8 mg; commercial) in THF (4 mL) was treated with NaH (49.6 mg) at 0° C. The mixture was stirred at rt for 3 days. The mixture was quenched with water and twice extracted with EA. The combined org. layers were dried over MgSO₄, filtered and concentrated under reduced pressure. The title compound was obtained, after purification by CC (CombiFlash, Hept to Hept/EA 2/1), as a colourless solid (179 mg; 63% yield).

MS1 (ESI, m/z): 230.01 [M+H⁺]; t_{R}=0.75 min.

### 6ii. 3-[6-(oxetan-3-yloxy)-pyridin-2-yl]-benzaldehyde

Starting from intermediate 6.i (160 mg) and 3-formylphenylboronic acid (112 mg; commercial) and proceeding in analogy to Example 1, step 1.ii, the title compound was obtained, after purification by CC (CombiFlash, Hept to Hept/EA 1/1), as a colourless oil (129 mg; 73% yield).

MS1 (ESI, m/z): 256.03 [M+H⁺]; t_{R}=0.84 min.

### 6.iii. 6-[(S)-5-(2-{3-[6-(oxetan-3-yloxy)-pyridin-2-yl]-benzylamino}-ethyl)-2-oxo-oxazolidin-3-yl]-4H-pyrido[3,2-b][1,4]oxazin-3-one

Starting from intermediate 6.ii (60 mg) and (S)-6-(5-(2-aminoethyl)-2-oxo-oxazolidin-3-yl)-2H-pyrido[3,2-b][1,4]oxazin-3(4H)-one (61.8 mg, prepared according to WO 2014/170821) and proceeding in analogy to Example 1, step 1.iii, the title compound was obtained, after purification by prep-HPLC (method 1), as a colourless solid (36 mg; 33% yield).

MS1 (ESI, m/z): 518.12 [M+H⁺]; t_{R}=0.67 min.

### Example 7: 6-{(S)-5-[2-({2-[6-(oxetan-3-yloxy)-pyrazin-2-yl]-pyridin-4-ylmethyl}-amino)-ethyl]-2-oxo-oxazolidin-3-yl}-4H-pyrido[3,2-b][1,4]oxazin-3-one

### 7.i. 2-(4-dimethoxymethyl-pyridin-2-yl)-6-(oxetan-3-yloxy)-pyrazine

Starting from the compound of Preparation BB3 (150 mg) and oxetan-3-ol (66 mg; commercial) and proceeding in analogy to Example 5, step 5.i, the title compound was obtained as a yellow solid (170 mg; 99% yield).

MS1 (ESI, m/z): 304.03 [M+H⁺]; t_{R}=0.75 min.

### 7.ii. 2-[6-(oxetan-3-yloxy)-pyrazin-2-yl]-pyridine-4-carbaldehyde

Starting from intermediate 7.i (150 mg) and proceeding in analogy to Example 4, step 4.ii, the title compound was obtained as an orange solid (153 mg; quant.).

MS1 (ESI, m/z): 257.87 [M+H⁺]; t_{R}=0.72 min.

### 7.iii. 6-{(S)-5-[2-({2-[6-(oxetan-3-yloxy)-pyrazin-2-yl]-pyridin-4-ylmethyl}-amino)-ethyl]-2-oxo-oxazolidin-3-yl}-4H-pyrido[3,2-b][1,4]oxazin-3-one

Starting from intermediate 7.ii (80 mg) and (S)-6-(5-(2-aminoethyl)-2-oxo-oxazolidin-3-yl)-2H-pyrido[3,2-b][1,4]oxazin-3(4H)-one (45.4 mg; prepared according to WO 2014/170821) and proceeding in analogy to Example 1, step 1.iii, the title compound was obtained, after purification by prep-HPLC (method 1), as a light yellow solid (16 mg; 20% yield).

MS1 (ESI, m/z): 520.10 [M+H⁺]; t_{R}=0.59 min.

### Example 8: 6-[(S)-5-(2-{3-[5-(oxetan-3-yloxy)-pyridazin-3-yl]-benzylamino}-ethyl)-2-oxo-oxazolidin-3-yl]-4H-pyrido[3,2-b][1,4]oxazin-3-one

### 8.i. 5-chloro-3-(3-[1,3]dioxolan-2-yl-phenyl)-pyridazine

A mixture of 3,5-dichloropyridazine (314 mg, commercial), 3-(1,3-dioxolan-2-yl)phenylboronic acid pinacol ester (552 mg, commercial), Pd(OAc)₂ (22.5 mg), dppf (57.2 mg) and Cs₂CO₃ (1.63 g) in dioxane (8 mL) and water (2 mL) was degassed for 10 min with N₂ and sealed in a Schlenk flask. The resulting dark brown suspension was stirred at 70° C. overnight. The mixture was cooled to rt, diluted with EA, filtered through Celite and concentrated under reduced pressure. The title compound was obtained, after purification by CC (Hept to Hept/EA 1/1), as a colourless solid (339 mg; 65% yield).

MS4 (ESI, m/z): 263.12 [M+H⁺]; t_{R}=0.75 min.

### 8.ii. 3-(3-[1,3]dioxolan-2-yl-phenyl)-5-(oxetan-3-yloxy)-pyridazine

Starting from intermediate 8.i (120 mg) and oxetan-3-ol (268 mg; commercial) and proceeding in analogy to Example 5, step 5.i. The title compound was obtained, after purification by CC (CombiFlash, Hept to Hept/EA 1/4), as a yellow oil (66.4 mg; 48% yield).

MS4 (ESI, m/z): 301.09 [M+H⁺]; t_{R}=0.65 min.

### 8.iii. 3-[5-(oxetan-3-yloxy)-pyridazin-3-yl]-benzaldehyde

Intermediate 8.ii (94.2 mg) was stirred in MeCN/water 1:1 (4.4 mL) in the presence of CBr₄ (208 mg) at 80° C. for 1 h. The mixture was cooled to rt, sat. aq. NaHCO₃ was added and the mixture was extracted with EA (3×). The combined org. extracts were dried over MgSO₄, filtered and concentrated under reduced pressure. The title compound was obtained as a black oil (226 mg; quant.).

MS4 (ESI, m/z): 257.12 [M+H⁺]; t_{R}=0.65 min.

### 8.iv. 6-[(S)-5-(2-{3-[5-(oxetan-3-yloxy)-pyridazin-3-yl]-benzylamino}-ethyl)-2-oxo-oxazolidin-3-yl]-4H-pyrido[3,2-b][1,4]oxazin-3-one

A solution of intermediate 8.iii (80.5 mg) and (S)-6-(5-(2-aminoethyl)-2-oxo-oxazolidin-3-yl)-2H-pyrido[3,2-b][1,4]oxazin-3(4H)-one (87.4 mg, prepared according to WO 2014/170821) in DCM (3.2 mL) and DMF (0.66 mL) was treated at rt with NaBH(OAc)₃ (200 mg). The reaction mixture was stirred at rt for 1 h. The mixture was quenched with a few drops of water and concentrated under reduced pressure. The residue was purified by prep-HPLC (method 1) and CC (DCM to DCM/MeOH, 9/1) to afford the title compound as a dark yellow solid (54 mg; 33% yield).

MS4 (ESI, m/z): 519.05 [M+H⁺]; t_{R}=0.57 min.

### Example 9: 6-[(S)-5-(2-{[3′-(oxetan-3-yloxy)-biphenyl-3-ylmethyl]-amino}-ethyl)-2-oxo-oxazolidin-3-yl]-4H-pyrido[3,2-b][1,4]oxazin-3-one

### 9.i. 2-(3′-bromo-biphenyl-3-yl)-[1,3]dioxolane

To a solution of 3-(1,3-dioxolan-2-yl)phenylboronic acid pinacol ester (1.72 g; commercial) in DME (12.5 mL) was sequentially added 1-bromo-3-iodobenzene (0.676 mL, commercial) and 2M aq. Na₂CO₃ (7.8 mL). The reaction mixture was degassed and PdCl₂(PPh₃)₂ (36.5 mg) was added and the mixture was stirred at 80° C. for 6 h. After cooling to rt, the mixture was diluted with EA and washed with sat. NaHCO₃, 1N HCl and brine. The residue was dried over MgSO₄, filtered and concentrated under reduced pressure. The title compound was obtained after purification by CC (CombiFlash, Hept to Hept/EA 5:1) as a colourless oil (400 mg; 25% yield).

MS4 (ESI, m/z): 304.82 [M+H⁺]; t_{R}=0.94 min.

### 9.ii. 2-[3′-(oxetan-3-yloxy)-biphenyl-3-yl]-[1,3]dioxolane

To a suspension of intermediate 9.i (150 mg), oxetan-3-ol (49.8 mg) and cesium carbonate (243 mg) in toluene (2.2 mL) under N₂ were added Pd₂(dba)₃ (11.3 mg) and tBuXPhos (13.4 mg) and was then heated at 90° C. overnight. EA was added to the orange solution and the mixture was washed with water. The aq. layer was extracted twice with EA. The combined org. layers were dried over MgSO₄, filtered and concentrated under reduced pressure. The title compound was obtained, after purification by CC (CombiFlash, Hept to Hept/EA 1/1), as a yellow oil (116 mg; 79% yield).

MS3 (ESI, m/z): 299.03 [M+H⁺]; t_{R}=1.01 min.

### 9.iii. 3′-(oxetan-3-yloxy)-biphenyl-3-carbaldehyde

Intermediate 9.ii (108 mg) was dissolved in water (1.5 mL) and THF (1.5 mL), PTSA (11.5 mg) was added and the mixture was heated at 60° C. for 2 h. EA was added and the solution was washed with sat. aq. NaHCO₃. The aq. layer was extracted with EA. The combined org. layers were dried over MgSO₄, filtered and concentrated under reduced pressure. The title compound was obtained as a light yellow oil (76 mg; quant.).

¹H NMR (CDCl₃) δ: 10.12 (m, 1H); 8.09 (t, J=1.5 Hz, 1H); 7.90 (dt, J=7.6, 1.3 Hz, 1H); 7.85 (ddd, J=7.7, 1.8, 1.2 Hz, 1H); 7.64 (t, J=7.6 Hz, 1H); 7.38-7.42 ((m, 1H); 7.25-7.28 (m, 1H); 7.01 (m, 1H); 6.72 (ddd, J=8.2, 2.5 Hz, 1H); 5.29-5.34 (m, 1H); 5.02-5.05 (m, 2H); 4.82-4.85 (m, 2H).

### 9.iv. 6-[(S)-5-(2-{[3′-(oxetan-3-yloxy)-biphenyl-3-ylmethyl]-amino}-ethyl)-2-oxo-oxazolidin-3-yl]-4H-pyrido[3,2-b][1,4]oxazin-3-one

Starting from intermediate 9.iii (30 mg) and (S)-6-(5-(2-aminoethyl)-2-oxo-oxazolidin-3-yl)-2H-pyrido[3,2-b][1,4]oxazin-3(4H)-one (28.3 mg; prepared according to WO 2014/170821) and proceeding in analogy to Example 1, step 1.iii, the title compound was obtained, after purification by prep-HPLC (method 1), as a colourless solid (12 mg; 24% yield).

MS1 (ESI, m/z): 517.13 [M+H⁺]; t_{R}=0.68 min.

### Example 10: 6-{(R)-5-[2-({4-[4-(oxetan-3-yloxy)-pyrimidin-2-yl]-pyridin-2-ylmethyl}-amino)-ethyl]-2-oxo-oxazolidin-3-yl}-4H-pyrido[3,2-b][1,4]oxazin-3-one

Starting from intermediate 4.ii (40 mg) and 6-[(R)-5-(2-amino-ethyl)-2-oxo-oxazolidin-3-yl]-4H-pyrido[3,2-b][1,4]oxazin-3-one (36 mg; prepared according to WO 2014/170821) and proceeding in analogy to Example 1, step 1.iii, the title compound was obtained, after purification by prep-HPLC (method 1), as a colourless solid (35 mg; 54% yield).

MS1 (ESI, m/z): 520.14 [M+H⁺]; t_{R}=0.60 min.

### Example 11: 6-{(S)-5-[2-({4-[4-(oxetan-3-yloxy)-pyrimidin-2-yl]-pyridin-2-ylmethyl}-amino)-ethyl]-2-oxo-oxazolidin-3-yl}-4H-pyrido[3,2-b][1,4]thiazin-3-one

Starting from intermediate 4.ii (40 mg) and 6-[(S)-5-(2-amino-ethyl)-2-oxo-oxazolidin-3-yl]-4H-pyrido[3,2-b][1,4]thiazin-3-one (38.4 mg; prepared according to WO 2014/170821) and proceeding in analogy to Example 1, step 1.iii, the title compound was obtained, after purification by prep-HPLC (method 1), as a light colourless solid (24 mg; 36% yield).

MS1 (ESI, m/z): 536.14 [M+H⁺]; t_{R}=0.63 min.

### Example 12: 6-{(S)-5-[2-({4-[4-(oxetan-3-yloxy)-pyrimidin-2-yl]-pyridin-2-ylmethyl}-amino)-ethyl]-2-oxo-oxazolidin-3-yl}-4H-benzo[1,4]thiazin-3-one

Starting from intermediate 4.ii (40 mg) and 6-[(S)-5-(2-amino-ethyl)-2-oxo-oxazolidin-3-yl]-4H-benzo[1,4]thiazin-3-one (38.3 mg; prepared according to WO 2010/041194) and proceeding in analogy to Example 1, step 1.iii, the title compound was obtained, after purification by prep-HPLC (method 1), as a colourless solid (21 mg; 32% yield).

MS1 (ESI, m/z): 535.14 [M+H⁺]; t_{R}=0.62 min.

### Example 13: 6-{5-[2-({4-[4-(oxetan-3-yloxy)-pyrimidin-2-yl]-pyridin-2-ylmethyl}-amino)-ethyl]-2-oxo-oxazol-3-yl}-4H-pyrido[3,2-b][1,4]oxazin-3-one

Starting from intermediate 4.ii (30 mg) and 6-(5-(2-aminoethyl)-2-oxo-oxazol-3(2H)-yl)-2H-pyrido[3,2-b][1,4]oxazin-3(4H)-one (33.1 mg; prepared according to WO 2014/170821) and proceeding in analogy to Example 1, step 1.iii, the title compound was obtained, after purification by prep-HPLC (method 1), as a yellowish solid (15 mg; 31% yield).

MS1 (ESI, m/z): 518.00 [M+H⁺]; t_{R}=0.61 min.

### Example 14: 6-{(S)-5-[2-({5-hydroxy-4-[6-(oxetan-3-yloxy)-pyrazin-2-yl]-pyridin-2-ylmethyl}-amino)-ethyl]-2-oxo-oxazolidin-3-yl}-4H-pyrido[3,2-b][1,4]oxazin-3-one

### 14.i. Rac-2-[5-benzyloxy-2-(tetrahydro-pyran-2-yloxymethyl)-pyridin-4-yl]-6-(oxetan-3-yloxy)-pyrazine

To a suspension of the compound of Preparation BB4 (650 mg), intermediate 1.i (381 mg) and K₂CO₃ (455 mg) in 1,4-dioxane (7.6 mL) and water (0.08 mL) under N₂ was added Pd(PPh₃)₄ (95.2 mg). The mixture was degassed and flushed with N₂ and stirred at 110° C. for 45 min. The mixture was allowed to cool to rt and partitioned between EA and water, the aq. layer was extracted with EA and the combined org. layers were washed with brine, dried over MgSO₄, filtered and concentrated under reduced pressure. The title compound was obtained, after purification by CC (CombiFlash, Hept to Hept/EA 0/1) and recrystallization from Hept/TBME, as off-white crystals (380 mg, 51% yield).

MS1 (ESI, m/z): 450.03 [M+H⁺]; t_{R}=0.87 min.

### 14.ii. {5-benzyloxy-4-[6-(oxetan-3-yloxy)-pyrazin-2-yl]-pyridin-2-yl}-methanol

Intermediate 14.i (355 mg) was suspended with water (2 mL) and THF (2 mL) and AcOH (6 mL) was added under ice cooling. The ice bath was removed and the mixture was allowed to stir at rt to 75° C. for 3 h. The mixture was cooled to 0-5° C., diluted with water and carefully basified with NaOH. The mixture was extracted twice with EA and the org. layers were washed with water, dried over MgSO₄, filtrated and concentrated under reduced pressure. The title compound was obtained as a beige solid (324 mg; quant.).

MS1 (ESI, m/z): 366.02 [M+H⁺]; t_{R}=0.67 min.

### 14.iii. 5-benzyloxy-4-[6-(oxetan-3-yloxy)-pyrazin-2-yl]-pyridine-2-carbaldehyde

Intermediate 14.ii (324 mg) was dissolved in MeCN (8 mL) and the mixture was heated to 65° C. At this temperature, manganese(IV) oxide (856 mg; commercial) was added and the mixture was stirred at 65° C. for 1.5 h. The hot mixture was filtered over a glass fiber filter. The filtrate was concentrated under reduced pressure to afford the title compound as a beige solid (250 mg, 78% yield).

MS1 (ESI, m/z): 364.00 [M+H⁺]; t_{R}=0.86 min.

### 14.iv. 6-{(S)-5-[2-({5-benzyloxy-4-[6-(oxetan-3-yloxy)-pyrazin-2-yl]-pyridin-2-ylmethyl}-amino)-ethyl]-2-oxo-oxazolidin-3-yl}-4H-pyrido[3,2-b][1,4]oxazin-3-one

Starting from intermediate 14.iii (250 mg) and (S)-6-(5-(2-aminoethyl)-2-oxooxazolidin-3-yl)-2H-pyrido[3,2-b][1,4]oxazin-3(4H)-one (165 mg; prepared according to WO 2014/170821) and proceeding in analogy to Example 1, step 1.iii, the title compound was obtained, after purification by CC (CombiFlash, 0-10% [DCM/MeOH 9/1+1% NH₄OH] in DCM), as an off-white foam (235 mg; 63% yield).

MS1 (ESI, m/z): 626.09 [M+H⁺]; t_{R}=0.71 min.

### 14.v. 6-{(S)-5-[2-({5-hydroxy-4-[6-(oxetan-3-yloxy)-pyrazin-2-yl]-pyridin-2-ylmethyl}-amino)-ethyl]-2-oxo-oxazolidin-3-yl}-4H-pyrido[3,2-b][1,4]oxazin-3-one

Intermediate 14.iv (235 mg) was suspended with MeOH (8 mL) and DCM (2 mL) and 10% Pd/C (69 mg) was added under N₂. The mixture was hydrogenated for 20 h. The mixture was diluted with 8 mL DCM, the Pd/C was filtered off via a 45 μm PTFE syringe filter and the mixture was concentrated under reduced pressure. The title compound was obtained, after recrystallization from TBME/MeOH, as yellow crystals (175 mg; 87% yield).

MS1 (ESI, m/z): 536.11 [M+H⁺]; t_{R}=0.58 min.

### Example 15: 6-{(S)-5-[2-({5-hydroxy-4-[4-(oxetan-3-yloxy)-pyrimidin-2-yl]-pyridin-2-ylmethyl}-amino)-ethyl]-2-oxo-oxazolidin-3-yl}-4H-pyrido[3,2-b][1,4]oxazin-3-one

### 15.i. Rac-2-[5-benzyloxy-2-(tetrahydro-pyran-2-yloxymethyl)-pyridin-4-yl]-4-(oxetan-3-yloxy)-pyrimidine

Starting from the compound of Preparation BB4 (966 mg) and 2-chloro-4-(oxetan-3-yloxy)-pyrimidine (350 mg, commercial) and proceeding in analogy to Example 14, step 14.i, the title compound was obtained, after purification by CC (CombiFlash, Hept to Hept/EA 0/1), as a brown oil (420 mg; 55% yield).

MS1 (ESI, m/z): 450.02 [M+H⁺]; t_{R}=0.81 min.

### 15.ii. {5-benzyloxy-4-[4-(oxetan-3-yloxy)-pyrimidin-2-yl]-pyridin-2-yl}-methanol

Starting from intermediate 15.i (420 mg) and proceeding in analogy to Example 14, step 14.ii, the title compound was obtained, after purification by CC (CombiFlash, Hept to Hept/EA 0/1), as a colourless solid (150 mg; 52% yield).

MS1 (ESI, m/z): 366.02 [M+H⁺]; t_{R}=0.64 min.

### 15.iii. 5-benzyloxy-4-[4-(oxetan-3-yloxy)-pyrimidin-2-yl]-pyridine-2-carbaldehyde

Starting from intermediate 15.ii (150 mg) and proceeding in analogy to Example 14, step 14.iii, the title compound was obtained as a yellowish solid (150 mg; quant.).

MS1 (ESI, m/z): 364.01 [M+H⁺]; t_{R}=0.81 min.

### 15.iv. 6-{(S)-5-[2-({5-benzyloxy-4-[4-(oxetan-3-yloxy)-pyrimidin-2-yl]-pyridin-2-yl-methyl}-amino)-ethyl]-2-oxo-oxazolidin-3-yl}-4H-pyrido[3,2-b][1,4]oxazin-3-one

Starting from intermediate 15.iii (150 mg) and (S)-6-(5-(2-aminoethyl)-2-oxooxazolidin-3-yl)-2H-pyrido[3,2-b][1,4]oxazin-3(4H)-one (115 mg; prepared according to WO 2014/170821) and proceeding in analogy to Example 1, step 1.iii, the title compound was obtained, after purification by CC (CombiFlash, 0-10% [DCM/MeOH 9/1+1% NH₄OH] in DCM), as a slightly yellowish solid (141 mg; 55% yield).

MS1 (ESI, m/z): 626.09 [M+H⁺]; t_{R}=0.68 min.

### 15.v. 6-{(S)-5-[2-({5-hydroxy-4-[4-(oxetan-3-yloxy)-pyrimidin-2-yl]-pyridin-2-ylmethyl}-amino)-ethyl]-2-oxo-oxazolidin-3-yl}-4H-pyrido[3,2-b][1,4]oxazin-3-one

Starting from intermediate 15.iv (141 mg) and proceeding in analogy to Example 14, step 14.v, the title compound was obtained, after purification by CC (CombiFlash, 0-10% [DCM/MeOH 9/1+1% NH₄OH] in DCM), as a slightly yellowish solid (10 mg; 8% yield).

MS1 (ESI, m/z): 536.19 [M+H⁺]; t_{R}=0.62 min.

### Example 16: 6-{(S)-5-[2-({3-Hydroxy-4-[6-(oxetan-3-yloxy)-pyrazin-2-yl]-pyridin-2-ylmethyl}-amino)-ethyl]-2-oxo-oxazolidin-3-yl}-4H-pyrido[3,2-b][1,4]oxazin-3-one

### 16.i. 3-Benzyloxy-4-bromo-pyridine-2-carbaldehyde

To a solution of 3-benzyloxy-4-bromo-pyridine-2-carboxylic acid methyl ester (322 mg, commercial) in DCM (5 mL) was added a solution of diisobutylaluminum hydride 1M (1.2 mL) in a dropwise manner at −78° C. The resulting pale orange solution was stirred at −78° C. for 45 min. A solution Rochelle's salts was added, the mixture warmed to rt and extracted with EA. The organic layer was dried (MgSO₄), filtered and concentrated under reduced pressure. The title compound was obtained, after purification by CC (CombiFlash, Hept to Hept/EA 3/1), as a pale yellow oil (208 mg, 71%).

¹H NMR (CDCl₃) δ: 10.13 (s, 1H), 8.42 (d, J=4.9 Hz, 1H), 7.80 (d, J=4.9 Hz, 1H), 7.54-7.56 (m, 2H), 7.41-7.46 (m, 3H), 7.28 (s, 1H).

### 16.ii. 3-Benzyloxy-4-bromo-2-dimethoxymethyl-pyridine

A solution of intermediate 16.i (208 mg), trimethyl orthoformate (0.103 mL) and p-PTSA (4.06 mg) in MeOH (5 mL) was stirred at 50° C. for 2 days. The mixture was cooled down to rt and sat. aq. NaHCO₃ and EA were added. The layers were separated and the aq. phase was extracted with EA (2×). The combined org. extracts were dried over MgSO₄, filtered and concentrated under reduced pressure. The title compound was obtained, after purification by CC (CombiFlash, Hept to Hept/EA 1/1), as a pale yellow oil (190 mg, 79%).

MS4 (ESI, m/z): 337.94 [M+H⁺]; t_{R}=0.84 min.

### 16.iii. 2-(3-Benzyloxy-2-dimethoxymethyl-pyridin-4-yl)-6-(oxetan-3-yloxy)-pyrazine

A mixture of intermediate 16.ii (103 mg), BB5 (278 mg) and K₂CO₃ (126 mg) in dioxane (4 mL) and water (1 mL) was degassed with N₂ for 15 min. Pd₂(dba)₃ (14.2 mg) and tricyclohexylphosphine (10.6 mg) were then added and the closed reaction flask was heated at 85° C. for 1 h. The mixture was cooled to rt, diluted with EA, filtered through Celite and concentrated under reduced pressure. The title compound was obtained, after purification by CC (CombiFlash, Hept to EA), as a pale yellow solid (106 mg, 85%).

MS4 (ESI, m/z): 410.19 [M+H⁺]; t_{R}=0.79 min.

### 16.iv. 3-Benzyloxy-4-[6-(oxetan-3-yloxy)-pyrazin-2-yl]-pyridine-2-carbaldehyde

A solution of intermediate 16.iii (106 mg) and PTSA (10.1 mg) in water (2 mL) and THF (2 mL) was heated at 65° C. for 8 h. After cooling to rt toluene (5 mL) was added, the resulting solution was concentrated under reduced pressure and used directly in the next step.

MS4 (ESI, m/z): 364.17 [M+H⁺]; t_{R}=0.82 min.

### 16.v. 6-{(S)-5-[2-({3-Benzyloxy-4-[6-(oxetan-3-yloxy)-pyrazin-2-yl]-pyridin-2-ylmethyl}-amino)-ethyl]-2-oxo-oxazolidin-3-yl}-4H-pyrido[3,2-b][1,4]oxazin-3-one

Starting from intermediate 16.iv (94.1 mg) and (S)-6-(5-(2-aminoethyl)-2-oxooxazolidin-3-yl)-2H-pyrido[3,2-b][1,4]oxazin-3(4H)-one (72.1 mg; prepared according to WO 2014/170821) and proceeding in analogy to Example 1, step 1.iii, the title compound was obtained, after purification by CC (CombiFlash, 0-10% [DCM/MeOH 9/1+1% NH₄OH] in DCM), as a yellow oil (103 mg; 64% yield).

MS4 (ESI, m/z): 625.95 [M+H⁺]; t_{R}=0.71 min.

### 16.vi. 6-{(S)-5-[2-({3-Hydroxy-4-[6-(oxetan-3-yloxy)-pyrazin-2-yl]-pyridin-2-ylmethyl}-amino)-ethyl]-2-oxo-oxazolidin-3-yl}-4H-pyrido[3,2-b][1,4]oxazin-3-one

Starting from intermediate 16.v (103 mg) and proceeding in analogy to Example 14, step 14.v, the title compound was obtained, after purification by CC (CombiFlash, 0-10% [DCM/MeOH 9/1+1% NH₄OH] in DCM), as a yellow solid (20 mg; 23% yield).

MS4 (ESI, m/z): 536.11 [M+H⁺]; t_{R}=0.60 min.

### Pharmacological Properties of the Invention Compounds

In Vitro Assays

Bacterial Growth Minimal Inhibitory Concentrations:

### Experimental Methods

Minimal Inhibitory Concentrations (MICs; mg/L) were determined in cation-adjusted Mueller-Hinton Broth by a microdilution method following the description given in “Methods for Dilution Antimicrobial Susceptibility Tests for Bacteria that Grow Aerobically”, Approved standard, 7¹ ed., Clinical and Laboratory Standards Institute (CLSI) Document M7-A7, Wayne, Pa., USA (2006).

Results:

All Example compounds were tested against relevant Gram positive and Gram negative bacteria. Typical antibacterial test results are given in Table 1 hereafter (MICs in mg/L). Staphylococcus aureus A798 and Acinetobacter baumannii T6474 are multiply-resistant strains (in particular quinolone-resistant), whereas Moraxella catarrhalis A894 and Enterococcus faecalis ATCC29212 are quinolone-sensitive strains.

TABLE 1

| | MIC for | MIC for | MIC for | MIC for |
|---|---|---|---|---|
| Example | S. aureus | E. faecalis | M. catarrhalis | A. baumannii |
| No. | A798 | ATCC 29212 | A894 | T6474 |
| 1 | ≤0.031 | 0.25 | ≤0.031 | 0.125 |
| 2 | ≤0.031 | 0.25 | ≤0.031 | 0.125 |
| 3 | 1 | 1 | 0.25 | 1 |
| 4 | ≤0.031 | 0.25 | ≤0.031 | 0.125 |
| 5 | 8 | 8 | 0.5 | 8 |
| 6 | ≤0.031 | 0.25 | ≤0.031 | 0.25 |
| 7 | ≤0.031 | 0.5 | ≤0.031 | 0.5 |
| 8 | 0.5 | 4 | 0.25 | 1 |
| 9 | 0.125 | 0.5 | ≤0.031 | 1 |
| 10 | 0.25 | 0.5 | 0.063 | 0.25 |
| 11 | ≤0.031 | 0.125 | ≤0.031 | 0.125 |
| 12 | ≤0.031 | 0.25 | ≤0.031 | 0.25 |
| 13 | ≤0.031 | 0.5 | ≤0.031 | 0.125 |
| 14 | 1 | 4 | 0.5 | 4 |
| 15 | ≤0.031 | 0.25 | ≤0.031 | 0.25 |
| 16 | 0.25 | 2 | 0.25 | 1 |
| Cipro | >32 | 0.5 | ≤0.063 | >16 |

Moreover, the antibacterial properties of the compound of Example 9 have been compared with those of the compound of Example 50 of WO 2014/170821. These two compounds both correspond to the structure

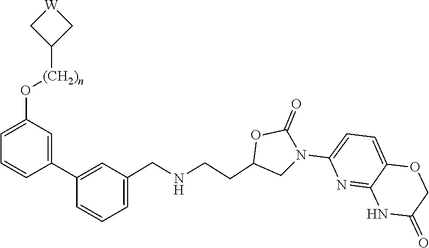

wherein either n is 0 and W is O (Example 9) or n is 1 and W is CH₂ (Example 50 of WO 2014/170821). The corresponding antibacterial test results are given in Table 2 hereafter (MICs in mg/L).

TABLE 2

| | MIC for | MIC for | MIC for | MIC for |
|---|---|---|---|---|
| Example | S. aureus | E. faecalis | M. catarrhalis | A. baumannii |
| No. | A798 | ATCC 29212 | A894 | T6474 |
| 9 | 0.125 | 0.5 | ≤0.031 | 1 |
| Ex. 50 of | 1 | 4 | 0.5 | >32 |
| WO 2014/ | | | | |
| 170821 | | | | |
| Cipro | >32 | 0.5 | ≤0.063 | >16 |

1. A compound of formula I 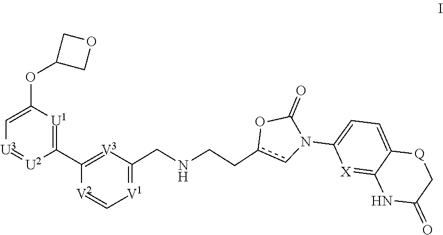
wherein
U¹ represents N or CH, U² represents N or CH, U³ represents N or CH, it being understood that at most two of U¹, U², U³ can represent N at the same time;
V¹ represents N or CH, V² represents N, CH or C(OH) and V³ represents N, CH or C(OH), it being understood that at most two of V¹, V² and V³ can represent N at the same time;
the dotted line “ [symbol] ” represents a bond or is absent;
X represents CH or N; and
Q represents O or S;
or a salt of the compound.

2. The compound of formula I according to claim 1, which is also a compound of formula I_{CE} 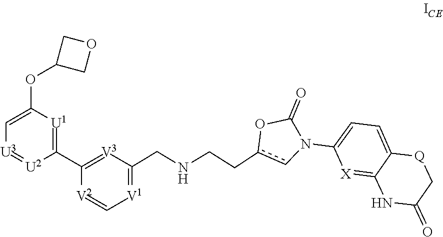
wherein
U¹ and U³ each represent N, U² represents CH, V¹ represents N and V² and V³ each represent CH, or
U¹ and U³ each represent CH, U² represents N, V¹ represents N and V² and V³ each represent CH, or
U¹ and U² each represent N, U³ represents CH, V¹ represents N and V² and V³ each represent CH, or
U¹ represents CH, U² and U³ each represent N, V¹ represents N and V² and V³ each represent CH, or
U¹ represents N and U², U³, V¹, V² and V³ each represent CH, or
U¹ and U³ each represent N, U² represents CH, V¹ and V³ each represent CH and V² represents N, or
U¹ represents CH, U² and U³ each represent N and V¹, V² and V³ each represent CH, or
U¹, U², U³, V¹, V² and V³ each represent CH, or
U¹ and U³ each represent N, U² represents CH, V¹ represents N, V² represents C(OH) and V³ represents CH, or
U¹ and U² each represent N, U³ represents CH, V¹ represents N, V² represents C(OH) and V³ represents CH;
the dotted line “ [symbol] ” represents a bond or is absent;
X represents CH or N; and
Q represents O or S;
or a salt of the compound.

3. The compound of formula I according to claim 1, wherein the dotted line “ [symbol] ” is absent;
or a salt of the compound.

4. The compound of formula I according to claim 1,
wherein the dotted line “ [symbol] ” represents a bond;
or a salt of the compound.

5. The compound of formula I according to claim 1, wherein X represents CH;
or a salt of the compound.

6. The compound of formula I according to claim 1, wherein X represents N;
or a salt of the compound.

7. The compound of formula I according to claim 6,
wherein Q represents O;
or a salt of the compound.

8. The compound of formula I according to claim 1, wherein
U¹ and U³ each represent N, U² represents CH, V¹ represents N and V² and V³ each represent CH, or
U¹ and U³ each represent CH, U² represents N, V¹ represents N and V² and V³ each represent CH, or
U¹ and U² each represent N, U³ represents CH, V¹ represents N and V² and V³ each represent CH, or
U¹ represents CH, U² and U³ each represent N, V¹ represents N and V² and V³ each represent CH, or
U¹ represents N and U², U³, V¹, V² and V³ each represent CH, or
U¹ and U³ each represent N, U² represents CH, V¹ and V³ each represent CH and V² represents N, or
U¹ represents CH, U² and U³ each represent N and V¹, V² and V³ each represent CH, or
U¹, U², U³, V¹, V² and V³ each represent CH; and
the dotted line “ [symbol] ” is absent;
or a salt of the compound.

9. The compound of formula I according to claim 1, wherein
U¹ and U³ each represent N, U² represents CH, V¹ represents N, V² represents C(OH) and V³ represents CH, or
U¹ and U² each represent N, U³ represents CH, V¹ represents N, V² represents C(OH) and V³ represents CH, or
U¹ and U³ each represent N, U² represents CH, V¹ represents N, V² represents CH and V³ represents C(OH); and
the dotted line “ [symbol] ” is absent;
or a salt of the compound.

10. The compound of formula I according to claim 1, wherein
U¹ and U³ each represent N, U² represents CH, V¹ represents N and V² and V³ each represent CH, or
U¹ and U² each represent N, U³ represents CH, V¹ represents N and V² and V³ each represent CH, or
U¹ represents N and U², U³, V¹, V² and V³ each represent CH, or
U¹ and U² each represent N, U³ represents CH, V¹ represents N, V² represents C(OH) and V³ represents CH; and
the dotted line “ [symbol] ” is absent;
or a salt of the compound.

11. The compound of formula I according to claim 1, which is selected from the following:
6-{(S)-5-[2-({4-[6-(oxetan-3-yloxy)-pyrazin-2-yl]-pyridin-2-ylmethyl}-amino)-ethyl]-2-oxo-oxazolidin-3-yl}-4H-pyrido[3,2-b][1,4]oxazin-3-one;
6-{5-[2-({4-[6-(oxetan-3-yloxy)-pyrazin-2-yl]-pyridin-2-ylmethyl}-amino)-ethyl]-2-oxo-oxazol-3-yl}-4H-pyrido[3,2-b][1,4]oxazin-3-one;
6-[(S)-5-(2-{[4-(oxetan-3-yloxy)-[2,4′]bipyridinyl-2′-ylmethyl]-amino}-ethyl)-2-oxo-oxazolidin-3-yl]-4H-pyrido[3,2-b][1,4]oxazin-3-one;
6-{(S)-5-[2-({4-[4-(oxetan-3-yloxy)-pyrimidin-2-yl]-pyridin-2-ylmethyl}-amino)-ethyl]-2-oxo-oxazolidin-3-yl}-4H-pyrido[3,2-b][1,4]oxazin-3-one;
6-{(S)-5-[2-({4-[5-(oxetan-3-yloxy)-pyridazin-3-yl]-pyridin-2-ylmethyl}-amino)-ethyl]-2-oxo-oxazolidin-3-yl}-4H-pyrido[3,2-b][1,4]oxazin-3-one;
6-[(S)-5-(2-{3-[6-(oxetan-3-yloxy)-pyridin-2-yl]-benzylamino}-ethyl)-2-oxo-oxazolidin-3-yl]-4H-pyrido[3,2-b][1,4]oxazin-3-one;
6-{(S)-5-[2-({2-[6-(oxetan-3-yloxy)-pyrazin-2-yl]-pyridin-4-ylmethyl}-amino)-ethyl]-2-oxo-oxazolidin-3-yl}-4H-pyrido[3,2-b][1,4]oxazin-3-one;
6-[(S)-5-(2-{3-[5-(oxetan-3-yloxy)-pyridazin-3-yl]-benzylamino}-ethyl)-2-oxo-oxazolidin-3-yl]-4H-pyrido[3,2-b][1,4]oxazin-3-one;
6-[(S)-5-(2-{[3′-(oxetan-3-yloxy)-biphenyl-3-ylmethyl]-amino}-ethyl)-2-oxo-oxazolidin-3-yl]-4H-pyrido[3,2-b][1,4]oxazin-3-one;
6-{(R)-5-[2-({4-[4-(oxetan-3-yloxy)-pyrimidin-2-yl]-pyridin-2-ylmethyl}-amino)-ethyl]-2-oxo-oxazolidin-3-yl}-4H-pyrido[3,2-b][1,4]oxazin-3-one;
6-{(S)-5-[2-({4-[4-(oxetan-3-yloxy)-pyrimidin-2-yl]-pyridin-2-ylmethyl}-amino)-ethyl]-2-oxo-oxazolidin-3-yl}-4H-pyrido[3,2-b][1,4]thiazin-3-one;
6-{(S)-5-[2-({4-[4-(oxetan-3-yloxy)-pyrimidin-2-yl]-pyridin-2-ylmethyl}-amino)-ethyl]-2-oxo-oxazolidin-3-yl}-4H-benzo[1,4]thiazin-3-one;
6-{5-[2-({4-[4-(oxetan-3-yloxy)-pyrimidin-2-yl]-pyridin-2-ylmethyl}-amino)-ethyl]-2-oxo-oxazol-3-yl}-4H-pyrido[3,2-b][1,4]oxazin-3-one;
6-{(S)-5-[2-({5-hydroxy-4-[6-(oxetan-3-yloxy)-pyrazin-2-yl]-pyridin-2-ylmethyl}-amino)-ethyl]-2-oxo-oxazolidin-3-yl}-4H-pyrido[3,2-b][1,4]oxazin-3-one;
6-{(S)-5-[2-({5-hydroxy-4-[4-(oxetan-3-yloxy)-pyrimidin-2-yl]-pyridin-2-ylmethyl}-amino)-ethyl]-2-oxo-oxazolidin-3-yl}-4H-pyrido[3,2-b][1,4]oxazin-3-one; and
6-{(S)-5-[2-({3-hydroxy-4-[6-(oxetan-3-yloxy)-pyrazin-2-yl]-pyridin-2-ylmethyl}-amino)-ethyl]-2-oxo-oxazolidin-3-yl}-4H-pyrido[3,2-b][1,4]oxazin-3-one;
or a salt of the compound.

12. A medicament comprising the compound of formula I as defined in claim 1, or a pharmaceutically acceptable salt thereof.

13. A pharmaceutical composition containing, as active principle, a compound of formula I as defined in claim 1, or a pharmaceutically acceptable salt thereof, and at least one therapeutically inert excipient.

14. A compound of formula I as defined in claim 1 for the prevention or treatment of a bacterial infection, or a pharmaceutically acceptable salt thereof.

15. The compound or pharmaceutically acceptable salt according to claim 14, which is for the prevention or treatment of a bacterial infection mediated by Staphylococcus aureus bacteria or Acinetobacter baumannii bacteria.

16. A medicament comprising the compound of formula I as defined in claim 11, or a pharmaceutically acceptable salt thereof.

17. A pharmaceutical composition containing, as active principle, a compound of formula I as defined in claim 11, or a pharmaceutically acceptable salt thereof, and at least one therapeutically inert excipient.

18. A compound of formula I as defined in claim 11 for the prevention or treatment of a bacterial infection, or a pharmaceutically acceptable salt thereof.

19. The compound or pharmaceutically acceptable salt according to claim 18, which is for the prevention or treatment of a bacterial infection mediated by Staphylococcus aureus bacteria or Acinetobacter baumannii bacteria.
